(19) United States
(12) Patent Application Publication
(10) Pub. No.: US 2024/0366215 A1
(43) Pub. Date: Nov. 7, 2024

(54) DEVICES, SYSTEMS, AND METHODS FOR PERFORMING A PROCEDURE WITH RESPECT TO TISSUE

(71) Applicant: Boston Scientific Scimed, Inc., Maple Grove, MN (US)

(72) Inventors: Vladimir Mitelberg, Austin, TX (US); John Mims, Austin, TX (US); Ryan Gilkey, Kyle, TX (US)

(73) Assignee: Boston Scientific Scimed, Inc., Maple Grove, MN (US)

(21) Appl. No.: 18/655,169

(22) Filed: May 3, 2024

Related U.S. Application Data
(60) Provisional application No. 63/541,183, filed on Sep. 28, 2023.
(60) Provisional application No. 63/464,007, filed on May 4, 2023.

(51) Int. Cl. A61B 17/04; A61B 17/00; A61B 90/00

(52) U.S. Cl. CPC A61B 17/0469; A61B 17/00234; A61B 90/08; A61B 2017/00296; A61B 2017/003; A61B 2017/00323; A61B 2017/0034; A61B 2017/00367; A61B 2017/047

(57) ABSTRACT

A movable device of a system for performing a medical procedure. The movable device has a reconfigurable portion capable of holding the movable device in a selected position with respect to other components of the system. The movable device may be moved with respect to other components of the system, and the reconfigurable portion may be bent or otherwise reconfigured with respect to the system to hold the movable device in a selected position. The movable device may be a tissue grasping device. The tissue grasping device may be formed to permit grasping and ungrasping of tissue without damaging the tissue. The tissue grasping device may include a tissue engagement element which may be formed from a helically laser cut tube.

### CROSS-REFERENCE TO RELATED APPLICATIONS

This application claims the benefit of priority under 35 U.S.C. § 119 to U.S. Provisional Application No. 63/541,183, filed Sep. 28, 2023, and U.S. Provisional Application No. 63/464,007, filed May 4, 2023, the entire disclosures of which are hereby incorporated by reference herein for all purposes. Any and all priority claims identified in the Application Data Sheet, or any correction thereto, are hereby incorporated by reference under 37 C.F.R. § 1.57.

### FIELD

The present disclosure relates to devices, systems, and methods including a movable device for performing a procedure with respect to tissue. More particularly, the present disclosure relates to a movable device of a system which has a reconfigurable portion capable of being reconfigured and engaged with respect to another component of the system, such as to maintain a position of the movable device, and associated methods. The present disclosure also relates to devices, systems, and methods for grasping tissue. More particularly, the present disclosure relates to devices, systems, and methods for grasping and ungrasping tissue within a body. Optionally, the device for grasping tissue has a reconfigurable portion capable of maintaining a selected grasped position of tissue.

### BACKGROUND

Various devices (e.g., instruments, tools, etc.) and systems for performing surgical operations or procedures are configured to be advanced, retracted, delivered, etc., through a tubular elongate member. For instance, various surgical procedures may be performed transluminally or endoscopically (with an endoscope), with a flexible tubular elongate member being extended through a natural orifice or cavity or passageway within a patient's body to avoid more invasive open surgery (in which the patient's body is cut open more extensively). Laparoscopic procedures may also utilize flexible tubular elongate member inserted through a small incision and through which devices and systems may be advanced, retracted, delivered, etc. The flexible tubular elongate member may be a medical scope, such as a pre-existing endoscope (e.g., a commercially available endoscope, or endoscope otherwise not customized for the devices or systems to be used therewith). Through-the-scope (“TTS”) endoscopic devices are passed through a working channel defined through the medical scope (e.g., through the flexible insertion portion of the medical scope) to perform a variety of diagnostic, therapeutic, etc., functions. Through-the-scope and other transluminally delivered devices include tissue graspers, tissue cutting devices, snares, suture devices, biopsy devices, etc. During transluminal and/or endoscopic procedures, it is common that the tubular elongate member (through which further devices are passed) is held and operated by a physician (“Primary User”), and one or more devices extended through the tubular elongate member are operated by a second physician, assistant, or procedure team member (“Secondary User”). During a procedure, the two Users must communicate to coordinate the positioning of the tubular elongate member and the device extended therethrough. As a result, the ongoing need to communicate and coordinate actions and activities may extend the time it takes to complete a procedure. Miscommunication or misunderstanding between the two Users may result in a suboptimal use of the tubular elongate member and the device used therewith, which may necessitate additional procedure steps or may result in unsatisfactory procedural outcome. It is with respect to these and other considerations that the present improvements may be useful. In addition, various improvements to tissue grasping devices and systems, are described herein which may be used independently or in conjunction with the improvements addressing the above-described challenges.

### SUMMARY OF THE DISCLOSURE

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

In accordance with various principles of the present disclosure, a system for performing a medical procedure within a patient includes a flexible elongate member insertable to a target site within patient; and a movable device movable with respect to the flexible elongate member. In some aspects, the movable device includes a reconfigurable portion which is reconfigurable by a user with respect to at least a portion of the flexible elongate member.

In some aspects, the reconfigurable portion is configurable with respect to the flexible elongate member to maintain a selected position of the movable device with respect to the flexible elongate member. In some aspects, the reconfigurable portion is bendable or otherwise deformable with respect to the flexible elongate member.

In some aspects, the flexible elongate member is a medical scope. In some aspects, the system further includes an auxiliary flexible tubular elongate member extendable along the medical scope and defining a lumen through which the movable device is movable. In some aspects, the movable device is movable through a working channel defined through the medical scope.

In some aspects, the movable device includes an operable component on a distal end thereof configured to engage tissue, and tissue engaged by the operable component exerts a distally-directed force on the movable device. In some aspects, the reconfigurable portion maintains the movable device with respect to the flexible elongate member and against a distally-directed force exerted thereon by tissue engaged by the movable device. In some aspects, the operable component is a helical tissue engagement element.

In some aspects, the reconfigurable portion is adjacent a user-engageable portion of the movable device.

In some aspects, the system further includes an additional device operable with the movable device. In some aspects, the additional device is a suturing device and the movable device comprise a tissue-engagement element.

In some aspects, the length of the reconfigurable portion is such that the same user that operates the movable device can also operate another device of the system, with the same hand or different hands. In some aspects, the system includes an endoscope, and the same user can operate the movable device with one hand and reconfigure the reconfigurable portion of the movable device with the same hand or a different hand to maintain the position of the movable device relative to the scope.

In accordance with various principles of the present disclosure, a system for performing a medical procedure within a patient includes a movable device movable with respect to another component of the system. In some aspects, the movable device includes a reconfigurable portion which is reconfigurable by a user with respect to at least the other component of the system; and an operable component comprising a tissue engagement element.

In some aspects, the tissue engagement element comprises a helical tissue-engaging element formed from a laser-cut tube.

In some aspects, the movable device includes a flexible control wire, at least a proximal portion thereof forming the reconfigurable portion of the movable device.

In some aspects, the operable component comprises a tissue-engagement element coupled to a distal end of the flexible control wire.

In some aspects, the system further includes an endoscope and a suturing device operatively associated with the endoscope, the movable device being movable with respect to the suturing device to draw tissue proximally into a path of a needle of the suturing device.

In some aspects, the length of the reconfigurable portion is such that the same user that operates the movable device can also operate another device of the system, with the same hand or different hands. In some aspects, the system includes an endoscope, and the same user can operate the movable device with one hand and reconfigure the reconfigurable portion of the movable device with the same hand or a different hand to maintain the position of the movable device relative to the scope.

In accordance with various principles of the present disclosure, a method of operating a system configured to perform a procedure within a patient includes moving a first device with respect to a component of the system; and reconfiguring a reconfigurable portion of the first device with respect to the component of the system to maintain the position of the first device in a selected position.

In some aspects, the first device is a tissue engagement element and the component is a suturing device; moving the first device further includes advancing the tissue engagement element into tissue, and retracting the tissue engagement element to draw the tissue in the path of the suturing device; and reconfiguring the reconfigurable portion of the first device includes engaging the reconfigurable portion with respect to a component of the system to maintain the position of the first device to maintain tissue in the path of the suturing device.

In some aspects, the method further includes reconfiguring the reconfigurable portion of the first device to release the movable device from its position engaged with a component of the system.

In some aspects, the same user operates the first device with one hand and maintains its position relative to another component of the system with the same hand or with the other hand.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1A is an elevational view of an example of an embodiment of a system with a movable device formed in accordance with various principles of the present disclosure and in a distally extended position.

FIG. 1B is an elevational view of a system with a movable device as in FIG. 1A, with the movable device in a proximally retracted position.

FIG. 1C is an elevational view of a system with a movable device as in FIG. 1B, with a reconfigurable portion of the movable device reconfigured to retain the position of the movable device with respect to another component of the system.

FIG. 2 is an elevational view of an example of an embodiment of a movable device formed in accordance with various principles of the present disclosure.

FIG. 2A is a detail view of area 2A in FIG. 2.

FIG. 3 is an elevational view of an example of an embodiment of a system as in FIGS. 1A-1C in a retroflexed configuration.

FIG. 4A is an elevational view of an example of an embodiment of a device and a movable device of a system formed in accordance with various principles of the present disclosure, with the movable device in a distally extended position.

FIG. 4B is an elevational view of an example of an embodiment of a device and a movable device of a system formed in accordance with various principles of the present disclosure, with the movable device in a proximally retracted position.

FIG. 5 is a perspective view of an endoscopic suturing system according an embodiment of the invention.

FIG. 6 is a proximal perspective view of a suturing device of the endoscopic suturing system of FIG. 5.

FIG. 7 is a side elevation view of a suturing device, of indefinite length, of the endoscopic suturing system of FIG. 5.

FIG. 8 is a perspective distal end view of a cap assembly attached at the distal end of an endoscope of the endoscope suturing system.

FIG. 9 is a perspective distal end view of cap assembly of the suturing device relative to an endoscope for the endoscopic suturing system.

FIG. 10 is a cross section view across line X-X in FIG. 5.

FIG. 11 is a schematic distal end view of the cap assembly of the suturing device relative to an endoscope for the endoscopic suturing system.

FIG. 12 is a view similar to FIG. 11 with the endoscope rotated into alignment.

FIG. 13 is a view similar to FIG. 9 with the endoscope shown properly oriented and advanced to the distal end of a cap assembly.

FIG. 14 is a schematic illustration of a tape applicator.

FIG. 15 is a schematic illustration of a taping system for securing a sheath of the suturing device relative to the endoscope.

FIG. 16 and FIG. 17 illustrate features of the tape of the tape system.

FIG. 18 and FIG. 19 are schematic illustrations of a retroflex system for the endoscopic system, as applied at the proximal handle of the system.

FIG. 20 is a schematic illustration of a detail of one embodiment of a lock for the retroflex system.

FIG. 21 is a schematic illustration of the endoscopic system subject to distal retroflex as applied by the retroflex system.

FIG. 22 is a schematic illustration of a detail of operation of the lock of FIG. 16.

FIGS. 23-27 illustrate use of the tape applicator to apply tape at the cap assembly to secure the cap assembly relative to the distal end of the endoscope.

FIG. 28 illustrates use of the tape along the sheath to secure the sheath to the endoscope.

FIGS. 29-33 illustrate use of the endoscopic system to endoscopically suture tissue.

FIG. 34 and FIG. 35 FIGS. 30 and 31 illustrate disassembly of the tape from about the cap assembly.

FIG. 36 illustrates disassembly of the sheath from the endoscope.

FIG. 37 and FIG. 38 illustrate examples of alignment of a cap assembly as in FIG. 11 and FIG. 12.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends, and “axial” generally refers to along the longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a channel, a cavity, or a bore. As used herein, a “lumen” or “channel” or “bore” or “passage” is not limited to a circular cross-section. As used herein, a “free end” of an element is a terminal end at which such element does not extend beyond. It will be appreciated that terms such as at or on or adjacent or along an end may be used interchangeably herein without intent to limit unless otherwise stated, and are intended to indicate a general relative spatial relation rather than a precisely limited location. As understood herein, “corresponding” is intended to convey a relationship between components, parts, elements, etc., configured to interact with or to have another intended relationship with one another. Finally, reference to “at” a location or site is intended to include at and/or about the vicinity of (e.g., along, adjacent, proximate, etc.) such location or site.

Various procedures performed within a patient's body may be performed with more than one medical instrument, device, tool, etc. (such terms being used interchangeably herein without intent to limit). For the sake of convenience, and without intent to limit, the present disclosure is written with reference to transluminal and/or endoscopic procedures in which devices and/or systems are passed through a patient's body via a natural orifice or small incision, and then within the patient's body, without cutting the patient open to access target sites within the patient's body. The devices and/or systems typically are delivered with the assistance of one or more delivery devices, such as a tubular elongate member with a lumen or working channel defined therein and through which at least one device or system may be advanced, retracted, or otherwise movably extended. It will be appreciated that reference may be made to lumens or working channels or the like interchangeably and without intent to limit. Moreover, it will be appreciated that reference may be generally made to movably extended to refer, generally, to various movements, including, without limitation, advancement or retraction (e.g., linearly, axially, longitudinally, etc.) and/or rotational movement through or within the lumen of a tubular elongate member. Finally, it will be appreciated that reference may be made interchangeably, and without intent to limit, to devices, instruments, tools, components, etc.

If one tubular elongate member is used with a system or method of the present disclosure, the one or more devices of the system, or one or more systems, may be extended through a common lumen therethrough. Optionally, at least one device may be extended along the exterior of a tubular elongate member. In some aspects, a tubular elongate member with more than one lumen is used with a system or method of the present disclosure, and the same or different of those lumens is used for the more than one device to be movably extended through the tubular elongate member. In some embodiments, a tubular elongate member is provided for each device. If more than two devices are used for the procedure, each device may be movably extended through its own lumen through a tubular elongate member, or two or more devices may be movable extended through a common lumen, or at least one device may be extended along the exterior of a tubular elongate member. In some embodiments, one tubular elongate member has more than one lumen, with one or more devices extending through one or more of the lumens. Optionally, one or more additional tubular elongate members may be provided for one or more additional devices to be used in performing the procedure. Typically, the more than one tubular elongate members are extended along one another, and optionally are coupled together, such as to be advanced together to a target site for the procedure. It will be appreciated that although the present disclosure generally describes two or more devices, various principles of the present disclosure may be applied to a single device used without additional devices. Moreover, it will be appreciated that various combinations of tubular elongate members, devices, systems, etc., other than those described herein may embody principles of the present disclosure, the present disclosure not being limited to examples of embodiments described herein.

The use of more than one instrument during a medical procedure typically requires coordination of the actions performed by or with the different instruments. It would be desirable for the same (i.e., a single) user/operator (e.g., medical professional) to be able to actuate, control, manipulate, operate, use, etc. (such terms and other grammatical forms thereof being used interchangeably herein without intent to limit) most if not all of the instruments while performing the procedure. In some aspects, at least one instrument is movable with respect to a handle of the system. In some instances, the system may include more than one handle (e.g., a handle for each instrument, and/or for each tubular elongate member advanced into the patient and controlled by the user). In some aspects, the movable instrument is moved by the operator thereof, with respect to various other components of the system, into a desired position. In accordance with various principles of the present disclosure, the instrument is positionable by the user, such as with respect to a handle of the system, to maintain the desired position of the instrument with respect to the other components or devices of the system. In some aspects, at least a portion of the movable instrument is reconfigurable to hold such reconfigurable portion with respect to the other components/devices of the system and in the desired position. In some aspects, the reconfigurable portion of the movable instrument is formable or deformable or shapeable (such terms, and other grammatical forms thereof, being used interchangeably herein without intent to limit). For instance, the reconfigurable portion may be formed into a shape which may hold or otherwise retain or anchor the movable instrument with respect to other components/devices of the system. In some aspects, the reconfigurable portion is formed into a shape, such as a hook, which may hold a position of the movable instrument with respect to a proximal end of the tubular elongate member (the proximal opening into the lumen through which the movable instrument extends). In some aspects, the reconfigurable portion is reconfigurable into its initial configuration, such as to undo the deformation thereof. In embodiments, the reconfigurable portion optionally is configurable to allow rotational movement of the movable instrument with respect to the other components/devices of the system while holding the movable instrument against longitudinal movement with respect to the other components/devices of the system.

In some embodiments, one of the more than one device of a system of the present disclosure is fixed with respect to a flexible elongate member such as a delivery device, and the movable instrument (e.g., such as described above) is axially translatable, and or rotationally movable with respect to the flexible elongate member. In other embodiments, both devices are movable. It will be appreciated that the present disclosure need not be limited in this regard.

In some aspects, the movable instrument is axially translatable to be advanced and/or retracted with respect to the delivery device, such as to be advanced distally out of a tubular elongate member and/or retracted into the tubular elongate member. In some aspects, at least a portion the tubular elongate member is capable of being articulated, bent, flexed, retroflexed, etc. (such terms, and other grammatical forms thereof, being used interchangeably herein without intent to limit). For instance, a distal portion of the tubular elongate member may be articulated to direct a movable instrument delivered by the tubular elongate member to a target site within the patient's body. In some aspects, it is desirable to limit the extent or distance the movable instrument is retracted so that the movable instrument does not extend too far into or proximal to a flexed section of the tubular elongate member, which may make later distal advancement from such position difficult. Various limiters, such as markings, may be provided to assist the user of the movable instrument in limiting proximal retraction thereof.

In some embodiments, the movable instrument is a tissue grasper. Various tissue graspers are known in the art. In some aspects, a tissue grasper formed in accordance with various principles of the present disclosure includes a helical element which may be rotated into tissue, and then axially proximally retracted (e.g., axially) to grasp and pull the tissue proximally. The present disclosure includes various improvements to tissue graspers to allow repeated use (grasping, pulling, releasing, etc.) thereof without damaging tissue or otherwise become inoperative.

Various embodiments of instruments movable with respect to components/devices of a system for performing a medical procedure, and associated methods, will now be described with reference to examples illustrated in the accompanying drawings. Reference in this specification to “one embodiment,” “an embodiment,” “some embodiments”, “other embodiments”, etc. indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that one or more of the features, structures, concepts, and/or characteristics described with reference to one embodiment can be combined with one or more of the features, structures, concepts, and/or characteristics of any of the other embodiments provided herein. That is, any of the features, structures, concepts, and/or characteristics described herein can be mixed and matched to create hybrid embodiments, and such hybrid embodiment are within the scope of the present disclosure. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. It should be appreciated that various dimensions provided herein are examples and one of ordinary skill in the art can readily determine the standard deviations and appropriate ranges of acceptable variations therefrom which are covered by the present disclosure and any claims associated therewith. The following description is of illustrative examples of embodiments only, and is not intended as limiting the broader aspects of the present disclosure.

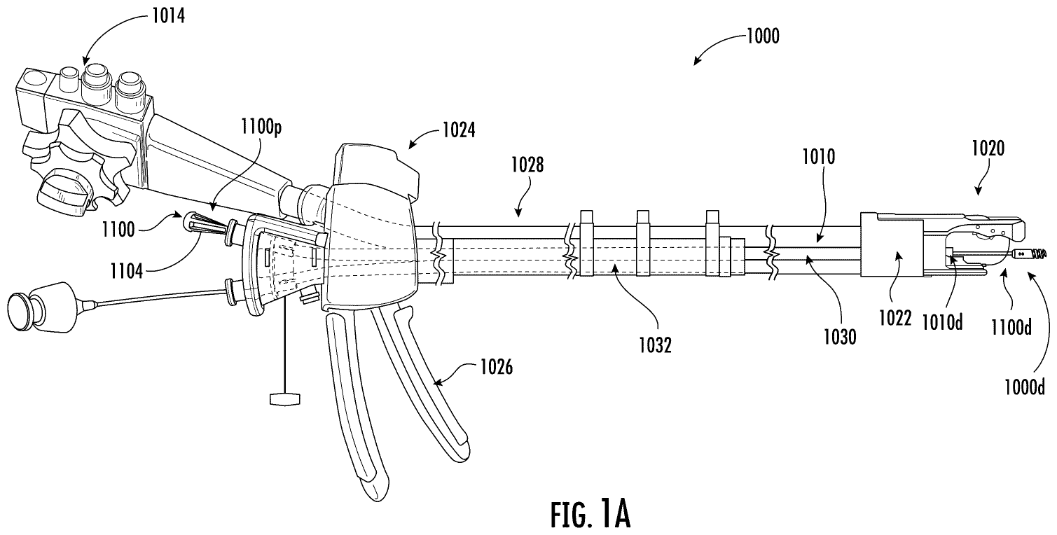

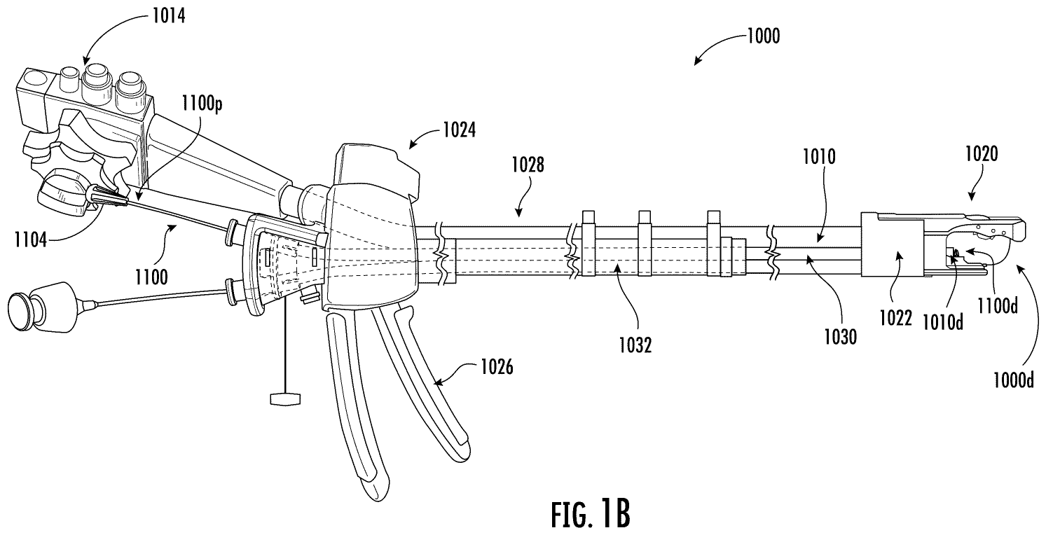

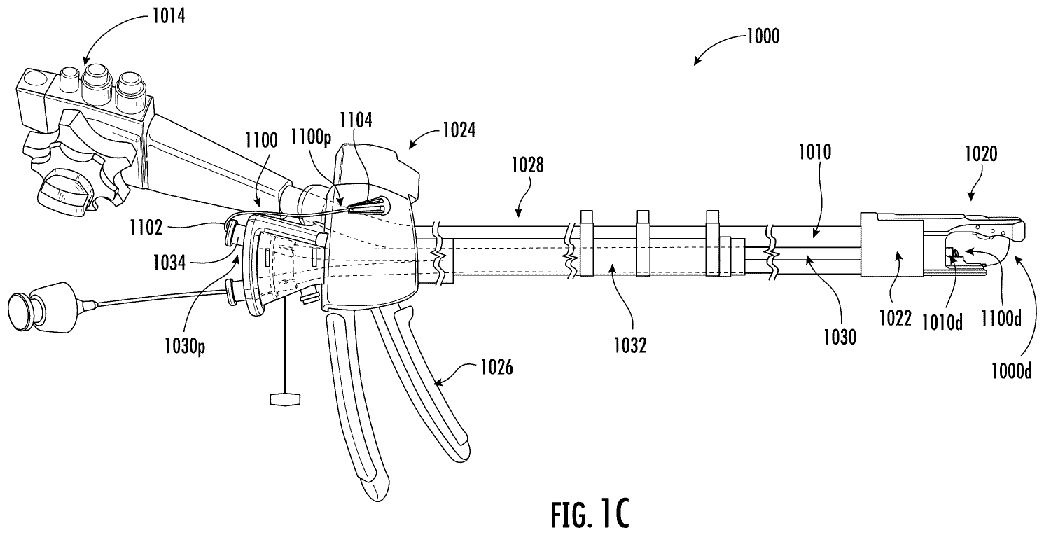

Turning now to the drawings, an example of an embodiment of a system 1000 for performing a procedure within a patient's body is illustrated in FIG. 1. The illustrated system 1000 includes a flexible elongate member 1010 which is insertable within a patient's body through a natural orifice or a small incision (e.g., laparoscopically), either through a lumen of a tubular elongate member (e.g., a tubular delivery device) or on its own, independent of being delivered with the assistance of and/or through another device. In the illustrated example of an embodiment, the flexible elongate member 1010 is an endoscope with one or more working channels defined therethrough. However, the present disclosure need not be limited in this manner.

In accordance with various principles of the present disclosure, the system 1000 further includes one or more devices associated with the flexible elongate member 1010 and configured to be advanced to a target site within a patient. In some aspects, the one or more devices are configured to be used to perform a procedure at a target site within a patient's body. In some aspects, the one or more devices are movable with respect to one another. In some embodiments, the one or more devices are insertable along or through (e.g., through a lumen or working channel of) or mounted with respect to (e.g., fixedly mounted with respect to) the flexible elongate member 1010. In the example of an embodiment illustrated in FIG. 1, at least one of the one or more devices is a device 1020 which is mounted with respect to a distal end 1010d of the flexible elongate member 1010. More particularly, in the illustrated example of an embodiment, the device 1020 is a suturing device mounted on an end cap 1022 which, in turn, is mounted on a distal end 1010d of the flexible elongate member 1010. Further details of an example of an embodiment of a device in the form of a suturing device mounted with respect to the flexible elongate member 1010 of a system 1000 formed in accordance with various principles of the present disclosure are provided below. It will be appreciated that the present disclosure is not limited to devices fixed with respect to the flexible elongate member 1010 and/or to suturing devices, the illustrated example of an embodiment simply being an example of one of various devices usable in a system 1000 of the present disclosure and/or with methods of the present disclosure. Furthermore, it will be appreciated that additional movable or fixed devices may be used with a system 1000 or method of the present disclosure, in addition to or instead of the device 1020.

In accordance with various principles of the present disclosure, the system 1000 includes a movable device 1100 which is movable with respect to other components of the system 1000, such as, without limitation, the flexible elongate member 1010. In the example of an embodiment illustrated in FIG. 1A, the movable device 1100 extends generally longitudinally along the flexible elongate member 1010. More particularly, in the example of an embodiment illustrated in FIG. 1A, the movable device 1100 extends along the exterior of the flexible elongate member 1010, rather than through a working channel (if present) of the flexible elongate member 1010. If the flexible elongate member 1010 has a working channel extending therethrough, such working channel may thus be available to accommodate additional devices of the system 1000 which may be extended therethrough. In some embodiments, such as in the example of an embodiment illustrated in FIG. 1A, FIG. 1B, and FIG. 1C, the movable device 1100 extends through a lumen of an auxiliary tubular elongate member 1030 extending along (e.g., longitudinally along) the flexible elongate member 1010. In some aspects, the lumen of the auxiliary tubular elongate member 1030 may serve as an auxiliary working channel to the working channel of tubular flexible elongate member 1010 (such as an endoscope). In some aspects, the auxiliary tubular elongate member 1030 extends through a lumen of a sheath 1032 which is held (e.g., mounted) with respect to the flexible elongate member 1010.

In accordance with various principles of the present disclosure, the movable device 1100 is movable with respect to one or more components of the system 1000 of the present disclosure. For instance, in the example of an embodiment illustrated in FIG. 1, FIG. 1A, and FIG. 1C, the movable device 1100 is at least axially movable with respect to the flexible elongate member 1010. For instance, as illustrated in FIG. 1A, the movable device 1100 is positioned with respect to the flexible elongate member 1010 so that the distal end 1100d of the movable device 1100 extends distally with respect to (e.g., from, out of, beyond, etc.) the distal end 1010d of the flexible elongate member 1010. As illustrated in FIG. 1B, the movable device 1100 may be moved proximally with respect to the flexible elongate member 1010 so that the proximal end 1100p of the movable device 1100 extends further proximally (e.g., relative to the position illustrated in FIG. 1A) with respect to the proximal end 1000p of the system 1000 than as illustrated in FIG. 1A. As may be appreciated, such proximal movement of the movable device 1100 retracts the distal end 1100d thereof with respect to the device 1020. It will be appreciated that the movable device 1100 may also be rotatable with respect to flexible elongate member 1010.

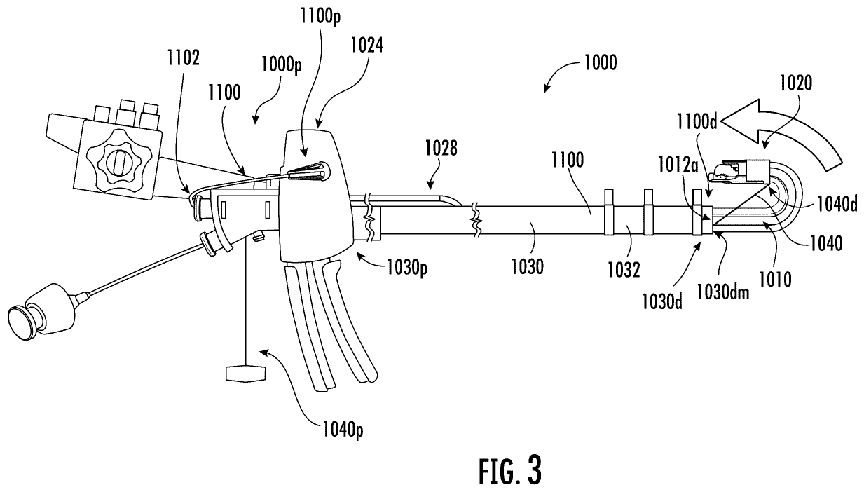

In some aspects, limiters, markers, markings, indicia, etc., (such terms being used interchangeably herein without intent to limit) are included along a proximal portion 1100p of the movable device 1100, such as to provide an indication to the operator of how far the movable device 1100 has been moved proximally. An example of an embodiment of a movable device 1100 formed in accordance with various principles of the present disclosure is illustrated in FIG. 3 with a plurality of indicia 1101 along at least a proximal portion 1100p thereof. The indicia 1101 may be raised, etched, engraved, colored, distinguishable from one another, etc., to facilitate use thereof during a procedure performed with the system 1000. In some aspects, the indicia indicate a distance the movable device 1100 has been moved proximally from another position.

The movable device 1100 optionally is usable with, in conjunction with, simultaneously with, sequentially with, etc., one or more of the other devices of the system 1000. For instance, the movable device 1100 optionally is usable with, in conjunction with, simultaneously with, sequentially with, etc., the device 1020 mounted with respect to the distal end 1010d of the flexible elongate member 1010 and/or the distal end 1000d of the system 1000, such as when the movable device 1100 is advanced distally beyond the distal end 1010d of the flexible elongate member 1010, such as illustrated in FIG. 1A.

In some aspects, a system 1000 formed in accordance with various principles of the present disclosure is capable of being retroflexed. An example of an embodiment of a system 1000 being retroflexed is illustrated in FIG. 3. In the illustrated example of an embodiment, the system 1000 includes an actuation component 1040 which may be referenced herein, for the sake of convenience and without intent to limit, as a pull string 1040. The pull string 1040 is an elongate element having sufficient strength to be actuated from a proximal end 1040p thereof to cause a distal end 1040d thereof to flex and/or retroflex one or more components of the system 1000, such as the distal end 1100d of the flexible elongate member 1010, and/or the distal end 1100d of the movable device 1100, and/or the distal end 1000d of the system 1000 generally. The pull string 1040 may be formed of a textile, polymer, or metal monofilament, multifilament, cable-stranded element, etc., having the appropriate strength, such as selected with reference to the longitudinal extent thereof, the flexibility of the system 1000 and/or one or more components thereof, etc. In some aspects, the movable device 1100 is movable with respect to one or more components of the system 1000 even when the system 1000 and/or components thereof are in a retroflexed configuration such as illustrated in FIG. 3. In some aspects, indicia 1101 may be used as retroflexion markers to indicate the distance the movable device 1100 is retracted proximally when the system 1000 is in a retroflexed configuration. Such indication or measurement is helpful for the user to be able to limit the extent the movable device 1100 is retracted proximally so that the movable device 1100 is not proximally retracted beyond a point at which it would be difficult to advance the distal end 1100d thereof distally through the retroflexed region of the system 1000 (e.g., through the auxiliary tubular elongate member 1030 through which the movable device 1100 extends).

During at least some uses of a movable device 1100 of the system 1000 of the present disclosure, it may be desirable to maintain a desired position of the movable device 1100 with respect to another component of the system 1000. For instance, if distal forces are applied to the distal end 1100d of the movable device 1100 during use thereof, it may be desirable to hold the movable device 1100 against being drawn or pulled distally. For instance, if the movable device 1100 is configured to grasp tissue and to pull/retract the tissue proximally, such as described in further detail below, the tissue may resist proximal grasping forces of the movable device 1100 and exert a distal force on the movable device 1100.

In accordance with various principles of the present disclosure, at least a portion of the movable device 1100 is reconfigurable to be maintained in a desired position with respect to another component of the system 1000. For instance, at least a portion 1102 of the movable device 1100 is reconfigurable to engage a component of the system 1000 and thereby be held with respect to such component. In some aspects, the reconfigurable portion 1102 is positioned along or adjacent a user-engageable portion along a proximal end 1100p of the movable device 1100. In the example of an embodiment illustrated in FIG. 1C, a reconfigurable portion 1102 of the movable device 1100 is provided adjacent a proximal end 1100p of the movable device 1100 and is reconfigurable with respect to a proximal end 1000p of the system 1000. For instance, in the example of an embodiment illustrated in FIG. 1C, the reconfigurable portion 1102 of the movable device 1100 may be bent or otherwise deformed with respect to a proximal end 1030p of an auxiliary tubular elongate member 1030 through which the movable device 1100 movably extends and/or another component of the system 1000, such as a component adjacent and/or proximal to a proximal end 1030p of the auxiliary tubular elongate member 1030. For instance, the reconfigurable portion 1102 may be hooked around a proximal end 1030p of the auxiliary tubular elongate member 1030. However, it will be appreciated that the bend in the reconfigurable portion 1102 need not be as extreme as illustrated in FIG. 1C. In some aspects, the reconfigurable portion 1102 may be reconfigured by pressing the reconfigurable portion 1102 with respect to the flexible elongate member 1010 (e.g., with respect to a handle 1014 thereof). Typically, the hand which operates the flexible elongate member 1010 (typically the left hand, particularly when the flexible elongate member 1010 is in the form of a medical scope) is used to reconfigure the reconfigurable portion 1102. The proximal end 1100p of the movable device 1100 may have a very low profile (even if provided with a handle 1104, such as described in further detail below), to be easily grabbed by slightly opening the hand operating the flexible elongate member 1010 when ready to reconfigure the reconfigurable portion 1102 of the movable device 1100. The user may then press the reconfigurable portion 1102 with respect to another component of the system 1000 to maintain the position of the movable device 1100 (e.g., to prevent the movable device 1100 from inadvertently changing its axial or rotational orientation).

In some embodiments, the auxiliary tubular elongate member 1030 is positioned within a lumen of a sheath 1032. In the example of an embodiment illustrated in FIG. 1C, the lumen of the auxiliary tubular elongate member 1030 and/or the sheath 1032 is accessible via a port 1034 positioned at a proximal end 1000p of the system 1000, and the reconfigurable portion 1102 of the movable device 1100 is bent or otherwise deformed and hooked around the port 1034, to be held in place with respect to the auxiliary tubular elongate member 1030, such as illustrated in FIG. 1C. In some aspects, the movable device 1100 remains rotatable with respect to another component of the system 1000 (e.g., an auxiliary tubular elongate member 1030 through which the movable device 1100 extends), even when held against longitudinal movement with respect to the other component of the system 1000. In some aspects, the reconfigurable portion 1102 is reconfigurable to release the movable device 1100 from the selected position in which it maintained with respect to another component of the system 1000, such as to allow relatively unrestrained movement of the movable device 1100 (e.g., axial and/or rotational movement not achievable when the movable device 1100 is in held in place as described above).

In some aspects, the movable device 1100 includes an operable component 1110 at a distal end 1100d of the movable device 1100 which may be configured to perform a medical procedure or task. In some aspects, movable device 1100 further includes a flexible elongate member 1120, with the operable component 1110 being provided along a distal end 1120d of the flexible elongate member 1120. In some aspects, a user-engaging element such as a handle 1104 is provided along the proximal end 1120p of the flexible elongate member 1120. In some aspects, the reconfigurable portion 1102 of the movable device 1100 is formed along or adjacent a proximal end/portion 1120p of the flexible elongate member 1120 (e.g., adjacent, such as slightly distal to, the handle 1104).

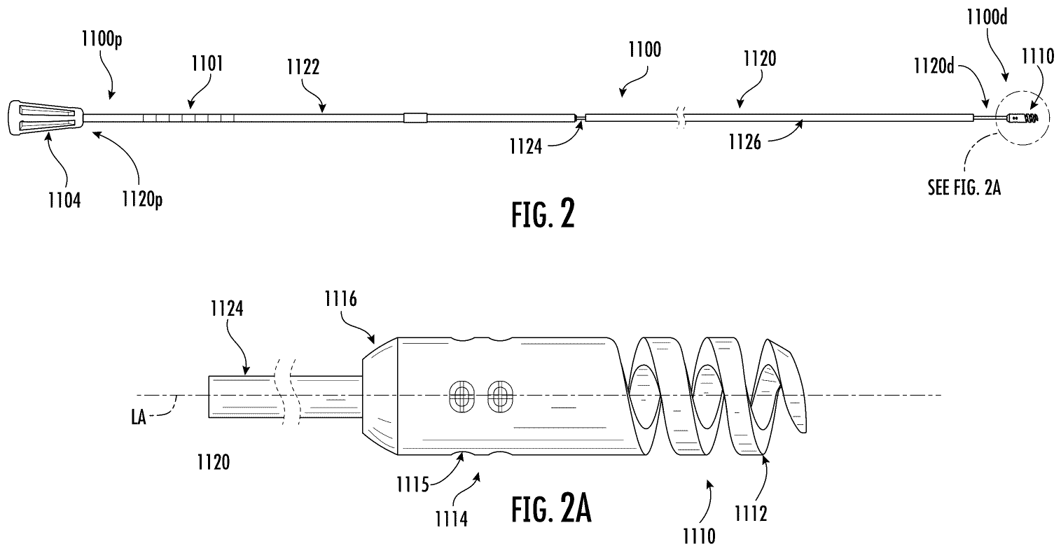

In some embodiments, such as illustrated in FIG. 2, the flexible elongate member 1120 includes a sheath 1122, a flexible control wire 1124 extending therethrough from the proximal end 1100p of the movable device 1100 to the distal end 1100d of the movable device 1100, and a bearing 1126. In some aspects, the handle 1104 is operatively coupled with the flexible control wire 1124. The sheath 1122, which may be referenced herein as a “control sheath”, may also be operatively coupled with the handle 1104. The control sheath 1122 may extend any length equal to at least the maximum amount that the operable component 1110 extends distally beyond the distal end 1010d of the flexible elongate member 1010 (in some embodiments, approximately 7 inches/17.8 cm). In some aspects, the control sheath 1122 is formed from a material that may be easily deformed by the user and may be easily moved between a first configuration to a second configuration. In the first configuration, the control sheath 1122 is generally axially elongated or in a straightened orientation which allows the flexible elongate member 1120 to move generally axially through the lumen of the auxiliary flexible tubular elongate member 1030 (e.g., to advance distally and/or to retract proximally) or through an instrument channel in the flexible elongate member 1010. In the second configuration, the control sheath 1122 is reconfigured, such as in a deformed/bent orientation that secures the flexible elongate member 1120 relative to the auxiliary flexible tubular elongate member 1030 and/or the flexible elongate member 1010. In some aspects, the control sheath 1122 can be made of a coil catheter wound from stainless steel wire or a thermoplastic extrusion material (e.g., a block copolymer such as formed of rigid polyamide blocks and soft polyether blocks, such as Pebax®, Nylon, etc.) with sufficient elastic deformation to allow the user to manipulate the control sheath 1122 to secure a selected/desired rotational and/or axial position of the movable device 1100 with respect to other components of the system 1000. Preferably, the position of the movable device 1100 may be secured while the user is holding and/or controlling other components of the system 1000. This allows the user to reconfigure the reconfigurable portion 1102 of the movable device 1100 as well as operate other components and/or devices and/or accessories of the system 1000 without the need for an assistant.

In some embodiments, the flexible control wire 1124 is formed from a material bendable/flexible material such as nitinol, or a multifilar torque wire wound from multiple strands of wire (e.g., metal wire, such as steel wire with or without nitinol wire) which can be easily deformed without plastic deformation or permanent set. In some aspects, the material of the flexible control wire 1124 is capable of translating axial movement and/or torque from the handle 1104 of the movable device 1100 to the operable component 1110 at the distal end 1100d of the movable device 1100. For instance, the material of the flexible control wire 1124 may be selected to transmit axial or rotational movement of the handle 1104 to an operable component 1110 in the form of a tissue-engagement element to engage and disengage tissue even when the movable device 1100/flexible elongate member 1120 is in a bent and/or tortuous and/or retroflexed configuration, such as described in further detail below.

In some embodiments, the bearing 1126 is configured and positioned with respect to the flexible control wire 1124 to facilitate rotation of the flexible control wire 1124 inside the bearing 1126 (e.g., and also within a lumen of the flexible elongate member 1010) especially when advanced through a tortuous body passage. Without a bearing 1126, the flexible control wire 1124 may tend to deflect and “whip” around, rather than rotate about its longitudinal axis (to advance/retract the tissue engagement element 1110 at the distal end thereof), thereby potentially adversely affecting rotational control of the flexible control wire 1124 and/or the tissue engagement element 1110. The bearing 1126 may also add sufficient column strength to at least the distal section of the flexible elongate member 1120, such as when the tissue engagement element 1110 is distally extended from the flexible elongate member 1010, so that the tissue engagement element 1110 can be advanced axially into tissue prior to being rotated into the tissue to achieve a secure initial depth engagement to achieve a high enough retention force with respect to tissue at the target site. It will be appreciated that the bearing 1126 may set a stiffness of at least a distal region of the flexible elongate member 1120. The stiffness set by the bearing 1126 may have a preferred range for the movable device 1100 to function as desired. If the stiffness imparted by the bearing 1126 is not high enough, then the flexible elongate member 1010 must be controlled very carefully in order to control movement of the movable device 1100. Moreover, if the stiffness is not high enough, the movable device 1100 generally performs very poorly tangentially (e.g., during tangential use of the flexible elongate member 1010, such as when a medical scope cannot be oriented perpendicular to the tissue at the target site), with the tissue engagement element 1110 potentially slipping along the tissue surface (e.g., slide along the mucosa of the tissue) rather than allowing the tip of the tissue engagement element 1110 to penetrate the tissue (e.g., to pierce the tissue upon being rotated with respect to the tissue). In some aspects, the stiffness imparted by the bearing 1126 preferably is sufficient to allow initial engagement of the tissue engagement element 1110 with tissue to pierce the tissue and penetrate into the tissue, and then to allow further distal force to be applied to the movable device 1100 (without the tissue engagement element 1110 sliding over the tissue), without causing the movable device 1100 to bend, to achieve a deeper tissue engagement. However, in contrast, if the stiffness imparted by the bearing 1126 is too high, the distal end 1100d may be so stiff as to pose a perforation risk (e.g., distal force may be transmitted too readily, causing the tissue engagement element 1110 to extend too deeply into, and potentially perforate, the tissue), and/or the stiffness may adversely affect overall maneuverability of the movable device 1100. In some aspects, it may be desirable for the movable device 1100 to be able to buckle (e.g., for the bearing 1126 to allow buckling of the movable device 1100 under certain conditions) if too much distal force is applied thereto, such as to protect the tissue contacted by the tissue engagement element 1110. In some aspects, the bearing 1126 is formed to stretch and/or compensate in length as the movable device 1100 is navigated through tortuous body passages. In some aspects, the bearing 1126 is formed so as not to take on a set/deformation when in a tight bending radius (e.g., in a particularly tortuous body passage, or in a high degree of retroflexion). As such, a bearing 1126 formed in accordance with various principles of the present disclosure is designed to work in the most extreme situations in which the movable device 1100 may be used. It will be appreciated that although the bearing 1126 is illustrated as being limited to a distal region of the movable device 1100, in some embodiments, the bearing 1126 may extend closer to proximal end 1100p of the movable device 1100. In some aspects, some clearance on either end of the movable device 1100 allows the bearing 1126 to “float” so it does not bind (e.g., with the tissue engagement element 1110 and/or with the handle 1104).

In some aspects, various devices of a system 1000 formed in accordance with various principles of the present disclosure have respective handles at respective proximal ends thereof which are manually graspable by the operator of the system 1000 to operate one or more of the devices of the system 1000. In accordance with various principles of the present disclosure, various handles may be associated with one or more components of the system 1000. The handles may be positioned at a proximal end 1000p of the system 1000 in sufficiently close proximity to allow the same to operate to selectively operate two or more devices of the system 1000, such as simultaneously and/or sequentially.

For instance, in the example of an embodiment of a system 1000 illustrated in FIGS. 1A-1C, a handle 1014 is provided at the proximal end 1010p of the flexible elongate member 1010 and may facilitate/control operation of the flexible elongate member 1010 by the operator of the system 1000. For instance, in the illustrated example of an embodiment, the flexible elongate member 1010 is an endoscope, and the handle 1014 thereof includes various control knobs and ports (e.g., for insertion of materials, tools, etc., into the working channel of the flexible elongate member/endoscope 1010). However, the present disclosure need not be limited in this regard.

In the example of an embodiment of a system 1000 illustrated in FIGS. 1A-1C, a handle 1024 is provided along the proximal end 1000p of the system 1000, and may be used to facilitate/control operation of the device 1020. For instance, in the illustrated example of an embodiment, the handle 1024 includes an actuator 1026 (e.g., a lever arm or the like) which is readily accessed and actuated by the operator's hand. In some aspects, a transmission 1028 is operatively coupled between the handle 1024 and the device 1020 for actuation by the handle 1024 to operate one or more components of the device 1020. For instance, the device 1020 may include one or more movable components, and the transmission 1028 may be operable by actuating (e.g., moving) the actuator 1026 to cause the transmission 1028 to move a component of the device 1020 for use in performing a procedure at a target site within the patient. More particularly, if the device 1020 is a suturing device, the actuator 1026 may cause the transmission 1028 to move a suturing needle to suture tissue at the target site, such as in examples of embodiments described in further detail below. However, the present disclosure need not be limited in this regard.

In the example of an embodiment of a system 1000 illustrated in FIGS. 1A-1C, a handle 1104 is provided at the proximal end 1100p of the movable device 1100 and may facilitate/control operation of the movable device 1100 by the operator of the system 1000. For instance, the handle 1104 may be shaped to facilitate axial translation and/or rotation of the movable device 1100 with respect to other components of the system 1000, such as in various manners described above. For instance, the handle 1104 may be shaped and/or configured (e.g., textured, ribbed, knurled, etc.) to allow for a level of rotational control necessary for a given medical procedure. In some aspects, the handle 1104 is sized, shaped, configured, and/or dimensioned so as not to interfere with the hand the user uses to reconfigure the movable device 1100 to be held with respect to another component of the system 1000.

Turning now to the example of an embodiment of a movable device 1100 illustrated in FIG. 2, the operable component 1110 of the movable device 1100, used to perform a task or procedure with respect to a target site, or otherwise usable with the system 1000 to perform a procedure, is a tissue engagement element 1110. At least with reference to the movable device 1100, terms such as engage, grasp, hold, etc., and other grammatical forms thereof, may be used interchangeably herein without intent to limit. In some aspects, the movable device 1100 may be referenced alternately herein as a tissue-engaging or tissue-grasping device. The tissue engagement element 1110 extends distally from a distal end 1100d of the movable device 1100. More particularly, the tissue engagement element 1110 extends distally from a distal end 1120d of a flexible elongate member 1120 of the movable device 1100. An optional handle 1104, such as a handle 1104 as described above, may be provided along a proximal end 1100p of the movable device 1100, such as along a proximal end 1120p of the flexible elongate member 1120.

In the example of an embodiment illustrated in FIG. 2, and the detail view of FIG. 2A (illustrating detail 2A in FIG. 2), the tissue engagement element 1110 includes a helical tissue-engaging portion 1112 extending from a body 1114. In some aspects, the tissue-engaging portion 1112 is formed with a sufficiently sharp/pointed tip to be able to pierce the targeted tissue. In some aspects, the tip of the tissue-engaging portion 1112 is formed/shaped by grinding, honing, laser cutting, etc., into a suitable configuration. In some aspects, the body 1114 includes one or more crimp zones 1115 facilitating crimping of the body 1114 onto a distal end 1120d of the flexible elongate member 1020 (e.g., onto a distal end of the control wire 1124). The helical configuration of the tissue-engaging portion 1112 allows rotation of the tissue engagement element 1110 in a tissue-engaging direction about the longitudinal axis LA of the tissue engagement element 1110 (such as may be determined by the direction of windings of the helix, as may be appreciated by those of ordinary skill in the art) to advance the tissue engagement element 1110 into tissue of a patient. Likewise, rotation of the tissue engagement element 1110 in an opposite, tissue-releasing direction about the longitudinal axis LA of the tissue engagement element 1110 (as determined by the direction of windings of the helix, as may be appreciated by those of ordinary skill in the art) withdraws the tissue engagement element 1110 from the patient's tissue.

In accordance with various principles of the present disclosure, the tissue engagement element 1110 is formed from a biocompatible material, such as a metal (e.g., medical grade stainless steel). In some aspects, the material of the tissue engagement element 1110 is coated with a biocompatible coating such as polytetrafluoroethylene (PTFE). In some aspects, the tissue engagement element 1110 may be formed from a metal wire (e.g., medical grade stainless steel, nitinol, titanium, or other formable non-medical grade metals) or metal additive manufacturing or by micro-molding a material such as a polymeric material (e.g., polyetheretherketone (PEEK)). In some aspects, the tissue engagement element 1110 is formed of a laser cut tube. In some aspects, the tube is cut to form helical windings with substantially constant spacings therebetween. The constant spacing between helical windings of the tissue engagement element 1110 may be selected to allow repeated grasping, release, and regrasping of tissue at the same or different locations within the patient. For instance, the tissue engagement element 1110 may be rotated in a first rotation to be advanced with respect to tissue to grasp the tissue. More particularly, rotation of the tissue engagement element 1110 advances the helical windings thereof into tissue, such that proximal, generally axial withdrawal of the movable device 1100 proximally withdraws the tissue in which the tissue engagement element 1110. Rotation of the tissue engagement element 1110 in the opposite direction withdraws the helical windings from the tissue. Because the windings are spaced from one another a substantially constant distance, the patient's tissue does not get pinched between the windings, and the tissue engagement element 1110 may be rotated out of tissue without damaging the tissue. The tissue may thereby be relatively atraumatically released from the previously-grasped tissue (compared to release of prior helical tissue engagement elements). The tissue engagement element 1110 may then be rotated in the tissue-engaging direction again to grasp the tissue at the same location or at a second, different location. However, it will be appreciated that variable spacing between the windings may be desirable for certain applications, the present disclosure not being limited in this regard.

In some aspects, at least a portion 1116 of a proximal end of the body 1114 of the tissue engagement element 1110 is chamfered to readily be retracted into the auxiliary tubular elongate member. In some aspects, the engagement of the chamfered portion 1116 with the auxiliary tubular elongate member 1030 provides a tactile indication to the user as the tissue engagement element 1110 is proximally retracted into and/or against the distal end 1030d of the auxiliary tubular elongate member 1030 through which the movable device 1100 movably extends.

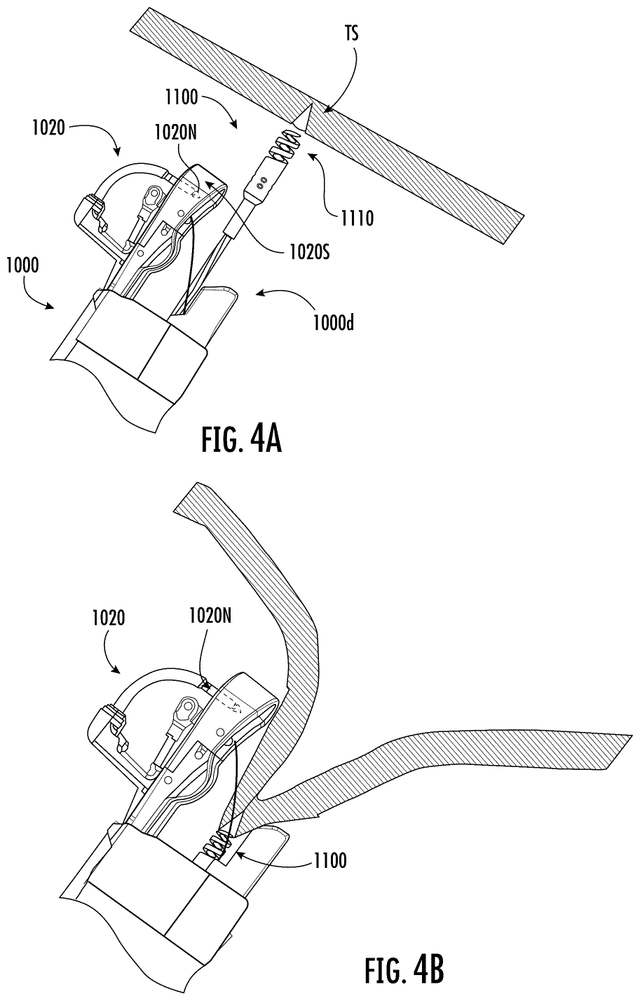

In some aspects, the movable device 1100 including a tissue engagement element 1110 facilitates performance of a procedure at a first location with a separate instrument. For instance, an example of an embodiment of a distal end 1000d of a system 1000 formed in accordance with various principles of the present disclosure is illustrated in FIG. 4A and FIG. 4B with a movable device 1100 in the form of a tissue grasping device, and a device 1020 in the form of a suturing device. As illustrated in FIG. 4A, distal advancement of the tissue engagement element 1110 engages the tissue engagement element 1110 with tissue at a target site TS within a patient. Proximal, generally axial, withdrawal of the movable device 1100 proximally withdraws the tissue at the target site TS into the pathway of a needle 1020N of the suturing device 1020 (shown shrouded below a shroud 1020S positioned to protect the patient's tissue from unintended contact by a sharp end of the needle 1020N), as illustrated in FIG. 4B. The needle 1020N may then be advanced through the tissue which has been grasped and proximally retracted into the needle pathway to advance a suture material, carried by the needle 1020N, through the tissue. Once the suture has been passed through the grasped tissue, the movable device 1100 may be distally extended and rotated in a tissue-releasing direction to be released from the tissue. The system 1000 may then be advanced to another location and the above described actions may be repeated. It will be appreciated that the present disclosure is not limited to suturing of tissue grasped by the movable device 1100. Other procedures may be performed with tissue grasped by a movable device 1100 in the form of a tissue grasper, such as may be repeated at first, second, third, fourth, etc., locations, within the patient's body. Additionally or alternatively, a first procedure is performed with the movable device 1100 at a first location, and the system 1000 is then moved to a second location at which a second procedure, different from the first procedure, is performed. The same or different procedures may be performed at third, fourth, etc., locations. It will be appreciated that the movable device 1100 may be operated in conjunction with another device either before, during, or after actions performed by the other device. In some aspects, the needle 1020N already has a suture operatively associated therewith when in the position illustrated in FIG. 4A and FIG. 4B, and thus moves the suture through the grasped tissue. Alternatively, the needle 1020N is initially moved through tissue grasped by the movable device 1100 (from the position illustrated in FIG. 4A and FIG. 4B) without a suture, to a position to grasp a suture operatively associated with the device 1020, and then back through the tissue and to the position illustrated in FIG. 4A and FIG. 4B, pulling the suture through the grasped tissue.

It will be appreciated that various additional devices other than the illustrated example of an embodiment of a movable device 1100 may be extended with respect to the flexible elongate member 1010, through a working channel of the flexible elongate member 1010 and/or through the same working channel of a delivery device, or separate working channels of a delivery device. In accordance with various principles of the present disclosure, any or all additional devices may have a proximal end adjacent the proximal end 1000p of the system 1000 and/or the proximal end 1100p of a movable device 1100 such as disclosed herein. The proximal end of any or all such additional devices may be positioned to be accessible by the operator of the system 1000 to be accessible while accessing a proximal end of another device at the proximal end 1000p of the system 1000. With the proximal end 1100p of the movable device 1100 held with respect to a component of the system 1000, the handle of an additional device may be grasped by the operator of the system 1000 and manipulated for use during the procedure being performed with the system 1000, leaving the movable device 1100 in a selected position as held by the reconfigurable portion 1102 thereof.

It will be appreciated that principles of the present disclosure may be applied to reconfiguring other types of movable devices (i.e., other than tissue grasping devices such as described above) with respect to a proximal end of a system such as, but not limited to, an endoscopic system, to resist a force exerted thereon. In some aspects, the proximal end of a movable device is reconfigurable so that the proximal end may be configured to be maintained in a selected position with respect to the proximal end of the system, released to allow the movable device to be moved again, and then optionally reconfigured again, such as to maintain a selected position with respect to the proximal end of the system (either the same as or different from any prior selected position). It will be appreciated that such reconfiguration of the proximal end of the movable device may be repeated two or more times, such as may be determined by the use of the movable device with respect to the patient. It will be appreciated that although the above example of an embodiment of a movable device with a reconfigurable portion is extended through an auxiliary flexible tubular elongate member, principles of the present disclosure may be applied to a device extendable through a working channel of a flexible elongate member such as a medical scope, and/or along the exterior of a flexible elongate member such as, but not limited to, a medical scope.

Various principles of the present disclosure such as those described above may be implemented in a variety of manners and/or applied to a variety of medical devices, examples of which will now be described. It will be appreciated that various methods described with respect to the described examples of embodiments may be applied to other embodiments, such as described more broadly/generically above.

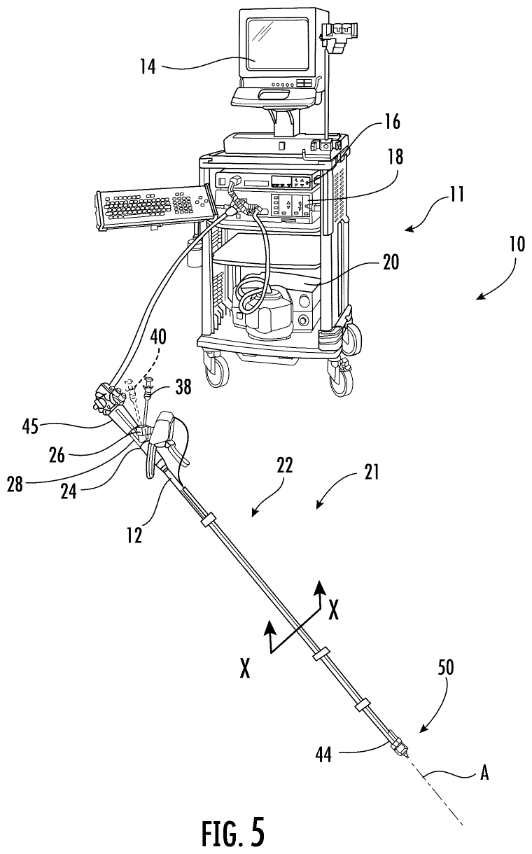

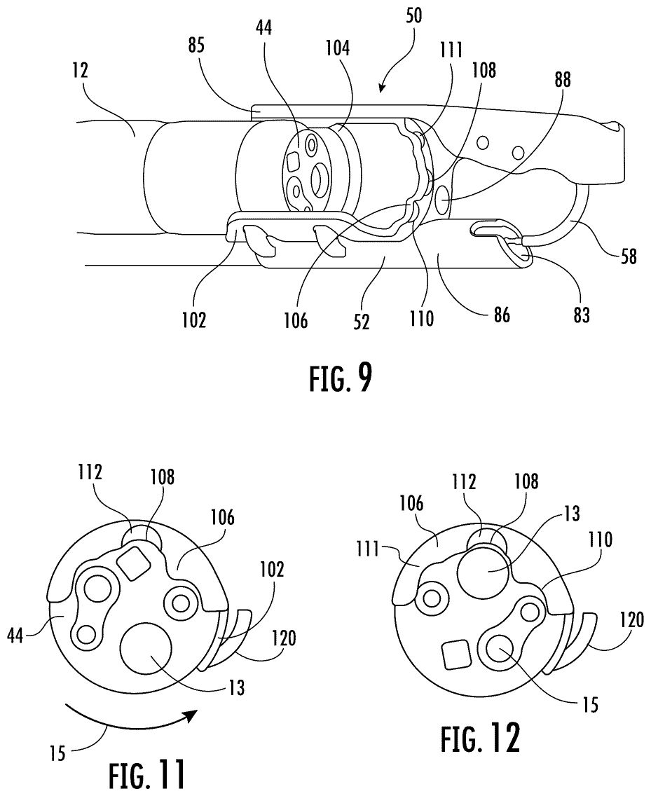

Referring to FIGS. 5-9, an endoscopic treatment system 10 includes an endoscope system 11 and an endoscopic suturing system 21. The endoscope system 11 includes an endoscope 12, a video display unit 14, an image processing device 16, a light source 18, and a suction device 20. In accordance with an embodiment, the endoscope 12 has a small profile, generally 5-10 mm in diameter. However, the size of the endoscope is not critical, and elements described herein can be adapted for endoscopes of other sizes. In the example of an embodiment shown, the endoscope 12 has a single instrument channel 13 and an optical lens 15 (FIG. 9). However, the endoscope 12 may have more than one instrument channel or no instrument channel all, as operation of the system does not necessarily require use of the instrument channel through the endoscope. The endoscope 12 includes a distal end 44 and a proximal end 45 and a longitudinal axis A extending therebetween.

The suturing system 21 includes a suturing device 22 (FIG. 7), a needle assembly 70 (FIG. 9) movable through tissue by the suturing device 22, and first and second devices 38, 40 used in association with the suturing device 22 (FIG. 9).

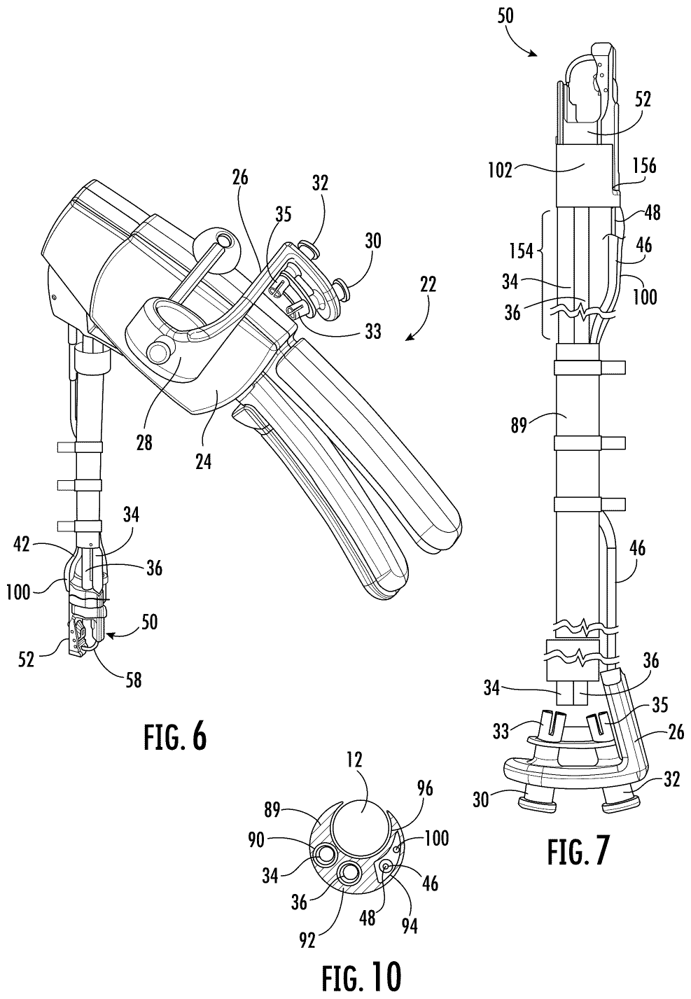

Referring to FIG. 5, FIG. 6, and FIG. 7, the suturing device 22 has a proximal operable handle 24 provided with a mounting bracket 26 and a collar 28 at which the handle is removably coupled to endoscope 12. The bracket 26 includes first and second instrument ports 30, 32 at which instruments can be received into first and second catheters 34, 36, respectively. First and second tubular connectors 33, 35 are aligned with the ports 30, 32 that couple the ports 30, 32 to the first and second catheters 34, 36.

A transmission assembly 42 includes a transmission sheath 46 and a transmission cable 48 displaceable within the transmission sheath 46, both coupled relative to the handle 24. The transmission sheath 46 is coupled relative to a first portion of the handle (i.e., a stationary member), and the transmission cable 48 is coupled to a second portion of the handle (i.e., a movable lever), such that when the handle 24 is operated the cable 48 is displaced within the transmission sheath 46.

The first and second catheters 34, 36 and the transmission assembly 42 extend from the proximal handle 24, along the outside of the endoscope 12, to a distal cap assembly 50. The distal cap assembly 50 is adapted to be mounted at the distal end 44 of the endoscope 12, and the handle 24 remotely operates the cap assembly 50 via the transmission assembly 42.

Referring to FIG. 9, the cap assembly 50 includes a mount 52, U-shaped support bracket 54 extending distally from the mount, and a needle arm 58 rotatably mounted on the bracket 54 with a first pin 60. A bell crank 64 is rotatably mounted at a second pin 66 on the support bracket 54 and engages the needle arm 58 at intermeshing gears (not shown). The distal end of the transmission cable 48 of the transmission assembly 42 is attached to the bell crank 64 at a clevis 68. When the transmission assembly 42 is operated by the handle 24, it results in rotation of the bell crank 64 and consequent rotation of the needle arm 58 between the open and closed positions.

The needle assembly 70 is coupled to a needle mount 83 at an end of the needle arm 58. The needle assembly 70 includes a tubular needle body 74, a needle tip 76, and suture 78 coupled to the needle body. The needle body 74 includes a side opening 80 through which the suture 78 extends, a first end 82 at which the needle assembly is coupled to the needle mount 83, and a second end 84 to which the tip 76 is coupled. The tip 76 defines a tissue-piercing taper. The suture 78 may be formed of any materials commonly available for surgical suture such as nylon, polyolefins, PLA, PGA, stainless steel, nitinol, and others. One suitable needle assembly is described in more detail in U.S. Pat. No. 9,198,562, which patent is hereby incorporated herein by reference in its entirety for all purposes.

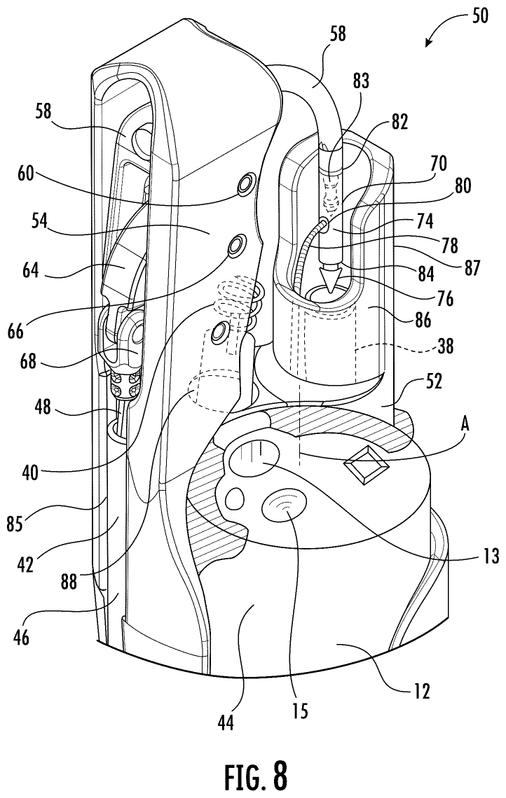

Referring to FIG. 8 and FIG. 9, the mount 52 of the cap assembly 50 includes a side recess 85 into which the transmission assembly 42 is received, and a first throughbore 86 and a second throughbore 88. The first throughbore 86 is positioned in alignment with both the needle mount 83 of the needle arm 58 and needle assembly 70 when the needle arm 58 is in the closed position. A tissue guide 87 extends distally on the mount 52 from over the first throughbore 86 and provides a surface on which to stabilize tissue as it is pierced by the needle assembly 70. The second throughbore 88 is positioned between the first throughbore 86 and the support bracket 54. More particularly, the axial center of the second throughbore 88 is positioned between the first throughbore 86 and the pin 60 (or axis) on which the end effector and/or assembly 58 rotates. The first and second throughbores 86, 88 may be parallel to each other and the longitudinal axis A of the endoscope, or the second throughbore 88 may be obliquely angled relative to the first throughbore 86 so as to direct the second device 40 at a particular orientation into the needle path, as described further below. The mount 52 is structured such that when the cap assembly 50 is coupled to the endoscope 12, as described below, the first and second throughbores 86, 88 are positioned radially outside the profile of the endoscope 12.

Referring to FIG. 7 and FIG. 10, the distal end of the first catheter 34 is fixed in the first throughbore 86, and its proximal end is coupled to a first connector 33 on the handle bracket 26. The distal end of the second catheter 36 is fixed in the second throughbore 88, and its proximal end is coupled the second connector 35 on the handle bracket 26. The first and second catheters 34, 36 may be discrete catheters or may be separate lumens of a common catheter. In a preferred embodiment, the catheters 34, 36 are discrete catheters covered in a preferably crescent moon shaped common sheath 89 along substantially all (i.e., at least 80 percent, and more preferably at least 90 percent) of their length. Alternatively, the common sheath 89 may extend along the entire length of the catheters 34, 36, a partial length thereof, or may be provided in sections along selected portions of the catheters 34, 36. In a preferred embodiment, the common sheath 89 is flexible, defines a lumen 90 for catheter 34, a lumen 92 for catheter 36, and a lumen 94 for both transmission assembly 42 and a retroflex pull string 100, the function of which is described below. Optionally, separate lumens can be provided for the transmission assembly 42 and the pull string 100. A concave recess 96 is provided along the length of the sheath 89 so that the sheath is preferably adapted to extend in close approximation to the exterior of an appropriately sized endoscope 12. In an embodiment, the sheath 89 is adapted to accommodate the endoscope such that the sheath preferably extends more than 180° about a circumference of, and thereby partially surrounds the exterior, of the appropriately sized endoscope 12.

Referring to FIG. 7 and FIG. 8, the first catheter 34 is adapted to receive a first device 38 that has a distal end effector that can receive and grasp the needle assembly 70. The second catheter 36 is adapted to receive a second device 40 that has a distal end effector that can engage tissue, and draw the tissue back into the path of the needle so that the tissue can be pierced by the needle assembly 70 as the needle assembly 70 is moved from the open to the closed position.

Referring to FIG. 9, the cap assembly 50 is secured to the distal end 44 of the endoscope 12 with a peripheral engagement structure that is adapted to be positioned about approximately 180°, and preferably slightly greater than 180°, around the circumference of the distal end of the endoscope 12. In one embodiment, the structure is a resilient clip 102 integrated at the proximal end of the mount 52. The clip 102 includes an opening 104 to allow the distal end 44 of the endoscope 12 access therethrough. The clip 102 may be formed from ABS plastic, other suitable plastics, elastic materials, as well as polymer-coated metals.

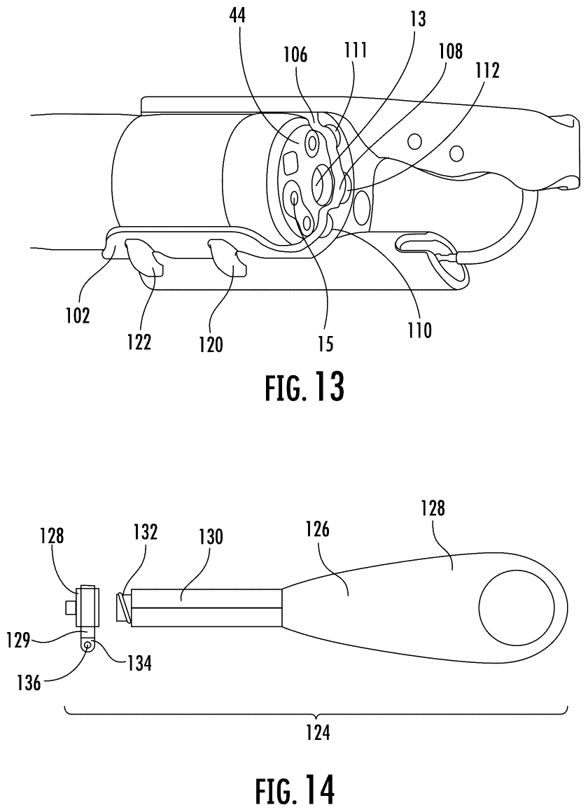

Turning to FIG. 9, FIG. 11, FIG. 12, and FIG. 13, a stop 106 is formed at the distal end of the clip 102. The distal end 44 of the endoscope 12 is adapted to pushed into the recess 104 of the clip 102 and seat against the stop 106. The stop 106 preferably includes a largest first clearance window, formed as a first recess 108 in the circumference of the stop 106 to permit pass through access to instrument channel 13 on various endoscopes of multiple manufacturers when the endoscope is properly rotationally aligned in the clip 102, and smaller second and third clearance windows, formed as second and third recesses 110, 111 approximately 60° on either side of the first recess 108, to prevent obstruction of the optical lens 15 and other working features of various endoscopes. The first recess 108 is located radially inward of the instrument channel lumen 88. In order to align the features of the endoscope relative to such first, second and third recesses 108, 110, 111, the stop 106 includes an indicia 112 relative to which the instrument channel 13 or other designated feature of an endoscope is rotationally aligned. For example, if an endoscope does not include an instrument channel 13 (as such is not required with the system described herein), the optical lens could be designated to be aligned with the indicia 112. In a preferred embodiment, the indicia 112 is a contrasting color relative to a color of a majority of the cap assembly which is provided about the first recess 108. With such rotational alignment of the designated endoscope feature relative to the indicia 112 (in the direction of arrow 115), the optical lens 15 of the endoscope is assured unobstructed view and appropriate orientation relative to the suturing arm 58, instrument channel lumen 88, and needle exchange lumen 86, regardless of the major manufacturer endoscope utilized with the system, without necessitating activating the endoscope. That is, the relative orientation, access, and field of view can be ascertain without requiring powering up the endoscope and previewing the field of view from the lens 13 on the display 14 in advance of the procedure. This significantly reduces preparation time prior to the procedure. The location of the first and second recesses 108, 110 and the indicia 112 relative to the suturing arm 58, instrument channel lumen 88, and needle exchange lumen 86, as well as areas which will be covered and blocked from view at the distal end of the endoscope, is based on analyses of scans of multiple distal ends of endoscopes.

Referring to FIG. 13, the resilient clip 102 further includes a plurality of longitudinally displaced (proximal and distal) integrated hooks 120, 122. In an embodiment, exactly two hooks are provided to the clip. The hooks are preferably identified with contrasting color relative to surrounding areas of the end cap and to a majority of the cap assembly. The hooks 120, 122 may also be numbered with indicia, e.g. “1” and “2”, according to the intended order of use, as further described below.

Turning to FIG. 14, the system also includes a tape applicator 124 for applying tape at each of the hooks 120, 122. The tape applicator 124 includes a handle 126 and a spool 128 of tape 129 on a distal end of the handle. The illustrated example of an embodiment of a handle 126 is preferably monolithic, including a wider proximal end 126p for stable gripping in a user's hand, and a narrower extension 130 for unobtrusively reaching about the distal end 44 of the endoscope 12 and cap assembly 50 during tape application. The extension 130 includes a spool mount 132. In a preferred embodiment the spool mount 132 is in the form of an external thread. The spool 128 is rotatably fixed on the spool mount 132 of the handle 126 during use. The spool 128 includes an internal thread (not shown) that allows the spool to be rotated onto the spool mount 132 in a rotational direction opposite the intended application of the tape, such that when tension is applied to the tape the spool is pulled in a direction that rotationally secures the spool relative to the mount. Other securing interfaces between the mount and spool can be used. The tape preferably has a non-adhesive free end 134 provided with an integrated loop 136 that is reinforced and sized for attachment to one of the hooks 120, 122. The reinforced integrated loop 136 is preferably provided in a color to facilitate locating it in the environment of an operating room. By way of example, the loop 136 is preferably opaque; and the loop preferably matches color with the hooks on the cap assembly. Both may be opaque white. In an embodiment, the loop 136 has a diameter of approximately 2.5 mm. The diameter of the spool 128 is preferably less than the diameter of the endoscope 12 to provide mechanical advantage when wrapping the tape 129 to secure the clip 102 and endoscope 12 relative to each other. In one embodiment, the spool has a diameter of 10 mm and a tape width of 5 mm. In an embodiment, except for the free end 134, the tape 129 has a consistent adhesion along its length and is primarily adapted to stick to the back of itself under tension, as well as both the endoscope 12 and the clip 102. Alternatively, the tape 129 can have a non-stick first portion for first wrapping about the endoscope, and a second adhesive portion for wrapping over and adhering to the back of the first portion and the back of the second portion.

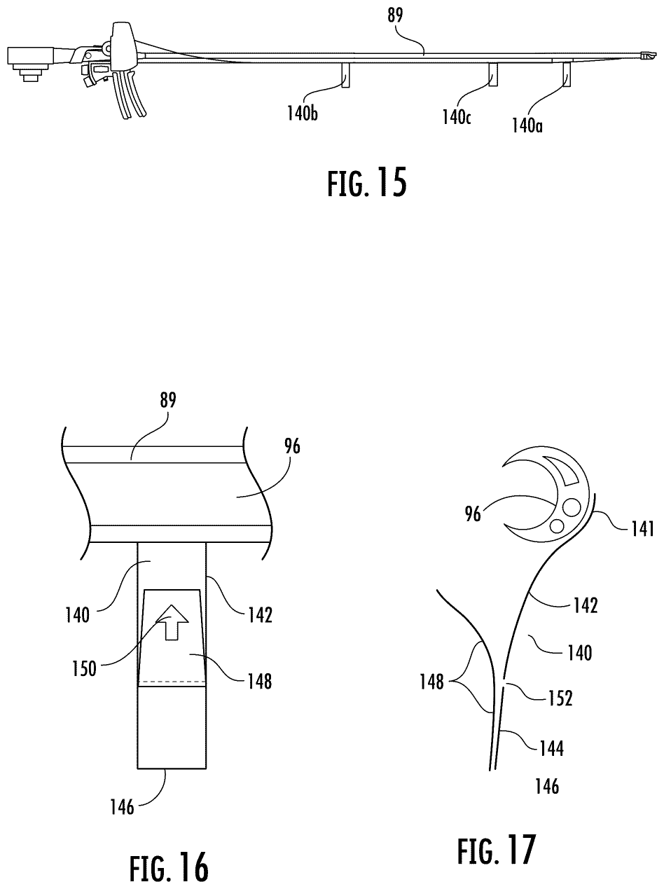

Turning now to FIG. 15, FIG. 16, and FIG. 17, a tape system is also provided for securing a portion of the sheath relative to the endoscope. The sheath 89 is provided with a plurality of strips of tape 140 pre-disposed along the length of the sheath 89. In a preferred embodiment, three strips of tape are used; a first strip 140a located at a distal end of the sheath, a second strip 140b located at a substantially central location along the length of the sheath, and a third strip 140c located between the first and second strips 140a, 140b and preferably closer to the first strip 140a. This limited use of tape and particular spacing has been shown to provide good stabilization of the sheath relative to the endoscope during use. That said, additional strips of tape can be pre-provided to the sheath and used in the manner described. In accordance with one aspect of the system, the strips of tape 140 have an adhered first portion 141 pre-applied to the sheath 89, a second portion 142 close to the sheath recess 96 that is non-sticky to prevent adhesion to the endoscope as the tape is wrapped around the endoscope, and a third portion 144 extending from the second portion 142 and to a free end 146 of the tape strip that is adhesive and adapted to adhere to the back of the tape when it is wrapped around the endoscope 12 and sheath 89. In accordance with another aspect, the tape strips 140 each include a pull off protective strip 148 that covers the second portion 144. The pull off protective strip 148 extends to cover the third portion 144 of the tape and is bent into an L-shaped extending tab between the free end 146 and the first portion 141. The tab 148 is preferably a high visibility color, contrasting with the sheath, e.g., white, and also preferably includes indicia, e.g., a laser-cut arrow 150, indicating the direction in which the tab 148 of the protective strip should be pulled to release the protective strip from the third portion to expose the adhesive (FIG. 16). In accordance with one aspect of the system, the strips 140 have at least a perforation 152 that permits breakaway disassembly after use of the system (FIG. 17). The perforation 152 is preferably provided at or near the junction of the second and third portions 142, 144 of the tape.

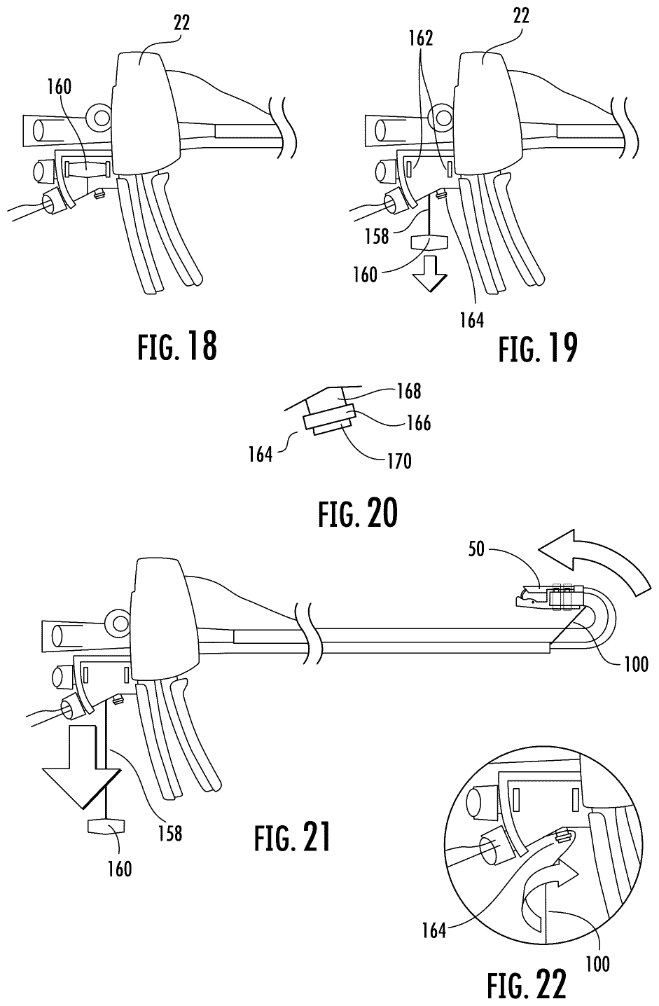

Turning to FIG. 7, FIG. 18, and FIG. 19, in accordance with another aspect of the system, the cap assembly 50 is adapted to retroflex relative to the sheath 89 via actuation from the proximal handle 24. The pull string 100, referenced above, extends from the proximal handle 24, through lumen 94 in the sheath 89, and exits the distal end of the sheath. A pull string 100 herein refers to a textile, polymer, or metal monofilament, multifilament, or cable stranded element. Between the distal end of the sheath and the end cap there is gap 154 (FIG. 7) exposing a length of the catheters 34, 36, the transmission assembly 42, and the pull string 100 outside the sheath 89 and to the cap assembly 50. The length of the gap 154 generally represents the difference in length between the endoscope 12, which has a longer first length, and the sheath 89, which has a shorter second length. In a preferred embodiment the exposed length of the gap 154 is less than 16 cm; more preferably the length of the gap is between 6 and 12 cm; even more preferably the length of the gap is between 7.5 and 10 cm; and most preferably the exposed length of the gap is 8.5 cm to provide optimal retroflexion, as described below. On the opposite side of the gap 154, the transmission assembly 42 is secured in the side recess 85 of the cap assembly 50 and the catheters 34, 36 are secured within the first and second throughbores 86, 88 (e.g., as illustrated in FIG. 8). Further, a distal end 156 of the pull string 100 is secured to the cap assembly 50. In one assembly, the distal end 156 is bonded into a hole (not shown) at the proximal end of the cap assembly 50. The proximal end 158 of the pull string 100 exits from the sheath and through an opening in the proximal handle 24 and is attached to a pull handle 160. A pull handle mount 162 is preferably formed on the proximal handle 24 and can secure the pull handle 160 via interference fit when not in use. A pull string lock 164 is provided on the proximal handle 24 to secure the pull string in a tensioned state. An example of an embodiment of a pull string lock 164 includes a resilient rubber washer 166 secured on a seat 168 and over a post 170 (FIG. 20). In use, when it is desirable to retroflex the cap assembly 50, the pull handle 160 can be released from the handle mount 162 and pulled to apply tension to the pull string 100. As tension is applied to the pull string 100, the pull string 100 is retracted across the gap 154 and through the lumen 94. As the pull string 100 is pulled across the gap 154, the cap assembly 150 is retroflexed to shorten the distance between the end of the sheath 89 and the location at which the distal end 156 of the string is attached to the cap assembly 50, as shown in FIG. 21. When a desired degree of retroflex is obtained, the tension on the pull string 100 may be temporarily maintained (and the retroflex configuration fixed) by wrapping the proximal end 158 of the pull string 100 around the post 170 between the washer 166 and the seat 168 (FIG. 22). Compression of the pull string 100 between the washer 166 and the seat 168 retains the pull string in position. The pull handle 160 can be placed back on the handle mount 162 to keep it out of the way (i.e., back to the position shown in FIG. 18) while other features of the proximal handle 24 and the endoscope 12 are used. Retroflex of the system may be used in combination with actuating retroflex of the endoscope. Then, when it is intended to release the retroflex condition at the distal end, the proximal end 158 of the pull string 100 is unwound from the pull string lock 164, and tension on the pull string is released. In such manner, retroflex of the system operates as an assist to the system attached to the endoscope and ensures that retroflex of the endoscope is carried out full in the manner intended. It is also recognized that this type of retroflex system, with or without assist from the endoscope, can be used in different endoscopic therapeutic and interventional systems, and is not limited to systems for suturing.

In light of the above, the suturing device may be prepared for use in conjunction with an endoscope as follows. The distal end 44 of the endoscope 12 is pushed into the resilient clip 104 until seated at or near the distal stop 106 (FIG. 9). Then, the endoscope and cap assembly 50 are coaxially rotated relative to each other until the instrument channel 13 of the endoscope is rotationally aligned with the indicia 112 at the distal stop 106 to ensure appropriate alignment between the two (FIG. 11 and FIG. 12). Then, if necessary, the endoscope 12 is pushed all the way into contact with the distal stop 106 (FIG. 13).

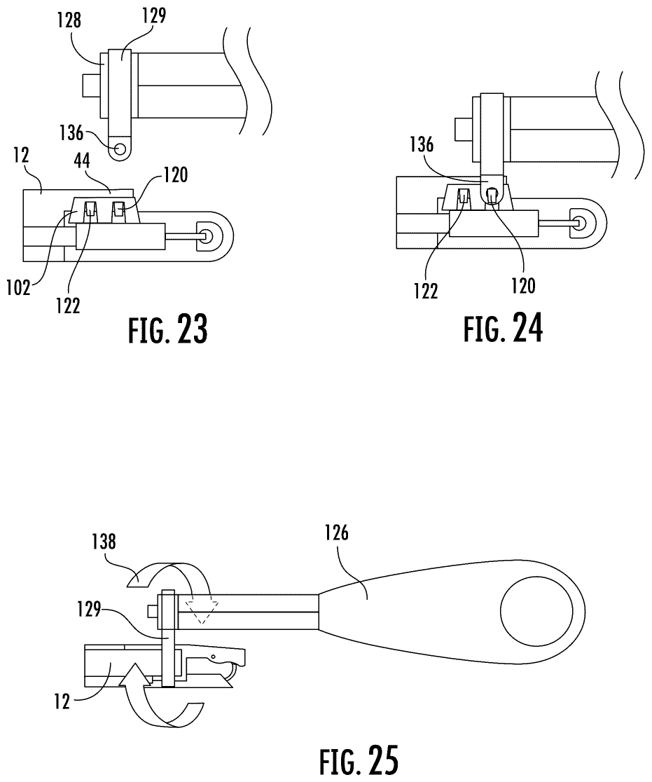

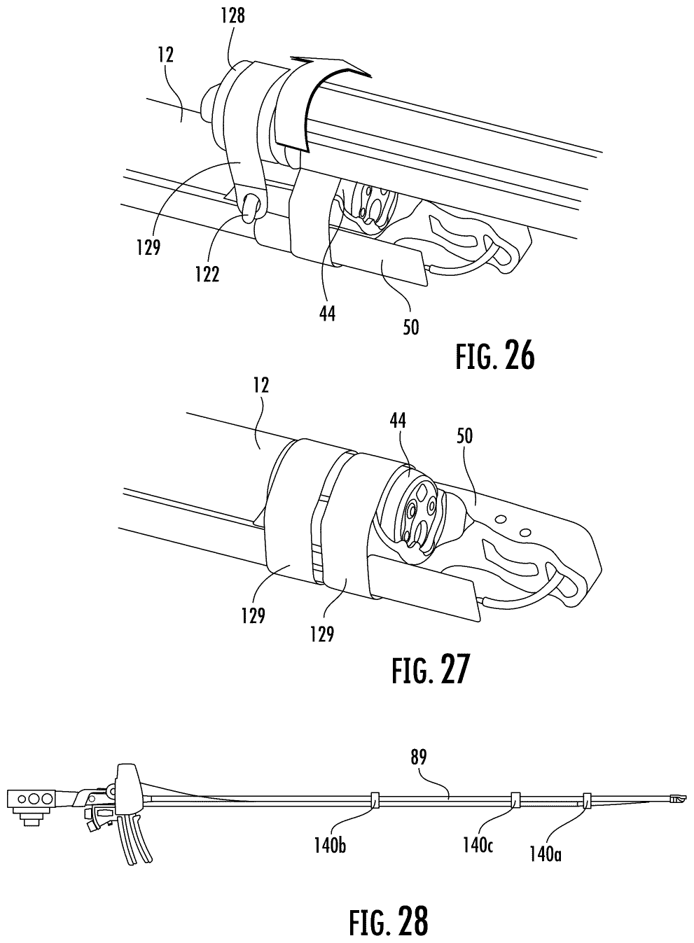

Then, referring to FIG. 23, FIG. 24, and FIG. 25, the loop 136 at the free end 134 of a first spool 128 of a tape applicator 124 is engaged over the distal first hook 120 and the handle 126 coupled to the spool 128 is maneuvered to wrap the tape 129 over a distalmost portion of the distal end 44 of the endoscope 12, over the clip 102, and back and around over the back of the tape 129 in a circular motion (arrow 138) one or more times to secure the endoscope relative to the clip. While wrapping, tension is applied to the tape. The relative diameters of the tape spool 128 and the endoscope 12 facilitate application of significant tension on the tape during wrapping of the endoscope with the tape. After the tape is applied at the first hook 120, the depleted spool 128 is removed from the spool mount 132, and a fresh spool 128 of tape is attached at the spool mount of the handle 126. Referring to FIG. 26 and FIG. 27, the tape 129 from the fresh spool 128 is similarly applied starting from the second hook 122 and over an adjacent area of the endoscope 12 until the distal end 44 of the endoscope 12 and cap assembly 50 are secured together.

Then, turning to FIG. 10, FIG. 16, FIG. 17, and FIG. 28, the endoscope is fully pressed into the recess 96 along the length of the sheath 89. Starting from the distal end of the system, and at strip of tape 140a, the tab 148 of the protective strip is pulled off to expose the adhesive portion 144, the tape 140 is pulled taut, the first portion 142 of the tape is wrapped around the endoscope 12, and the second adhesive portion 144 of the tape is adhered to the sheath on the opposite side of the recess 96 and then extended over the tape again to secure the endoscope thereat. The process is repeated along the length of the sheath for remaining strips 140c, then 140b, to properly secure the sheath relative to the endoscope. This longitudinally secures the sheath 89 to the exterior of the endoscope 12 for in vivo operation.

The collar 28 is properly positioned at the proximal handle 45 of the endoscope 12. The first device, a needle capture instrument 38 loaded with a needle assembly 70, is advanced through the first port 32, into the first lumen 34 and to the cap assembly 50. Suitable needle capture devices 38 are described in detail in U.S. Pat. No. 8,679,136, which patent is hereby incorporated herein by reference in its entirety for all purposes. The needle assembly 70 is loaded onto the needle arm 58, with the suture 78 extending parallel to the needle capture instrument 38 within the first lumen 34.

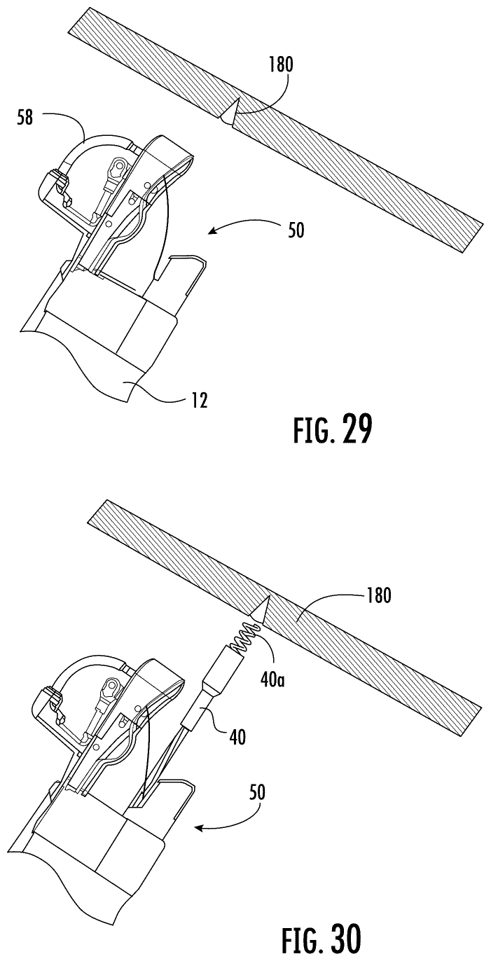

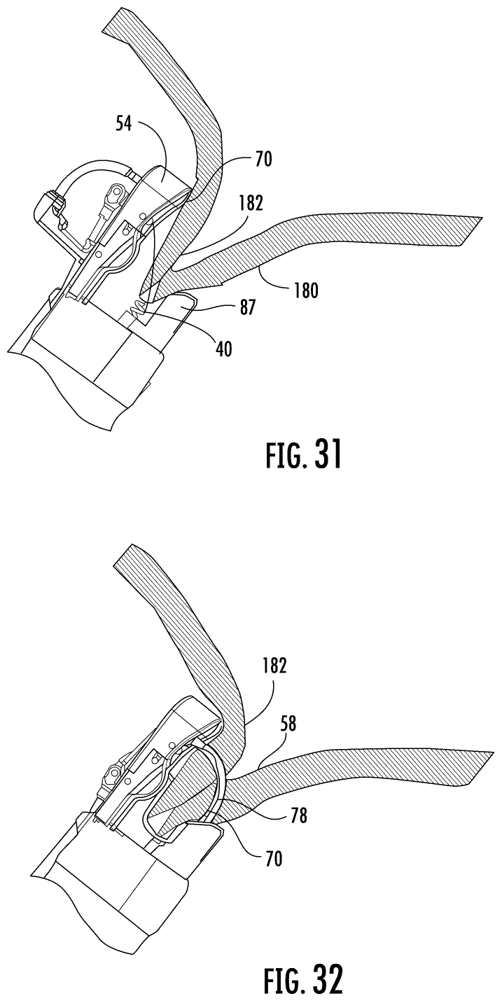

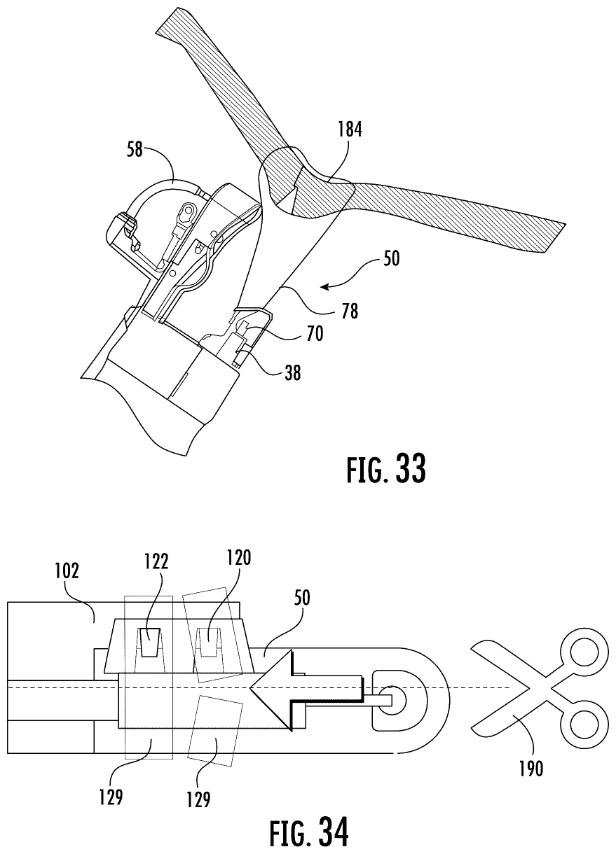

With reference to FIG. 15 and FIG. 29, the distal end of the endoscope 12 and cap assembly 50 of the suturing device 22 are advanced into a natural orifice of a patient, optionally through a guide tube (not shown), and approached to target tissue 180. If at any point it is necessary to reorient the endoscope 12 via retroflexion, the cap assembly 50 can be retroflexed as well, as described above, to aid and/or further direct the distal end of the endoscope and the cap assembly to the intended target tissue. Once adjacent the target tissue 180, the handle 24 of the suturing device 22 is operated to move the needle arm 58 into the open position, as shown in FIG. 28. Turning to FIG. 30, the end effector of the second device, e.g., a tissue retractor 40 having a helical coil 40a at its distal end, is advanced through the second port 32, into the second catheter 36 (FIG. 7) and out the second throughbore 88 (FIG. 8), and beyond the cap assembly 50. Suitable tissue retractor instruments are described in detail in U.S. Pat. No. 11,083,364, which patent is hereby incorporated herein by reference in its entirety for all purposes. Other tissue retractors, including forceps, may also be used. The helical coil 40a is operated to engage target tissue 180. The tissue retractor 40 is withdrawn to draw the tissue 180 against the tissue guard 87 and into a tissue fold 182 located within the path of the needle assembly 70; i.e., between the bracket 54 and needle guide 87, as shown in FIG. 31. The orientation of the second throughbore 88, either parallel or obliquely angled relatively to the first throughbore 86, is adapted to guide the tissue retractor to engage and retract tissue into the needle path. The handle 24 is then operated to move the needle arm 58 into the closed position, thereby piercing the tissue fold 182 and passing the needle assembly 70 with suture 78 through the tissue fold during the movement (FIG. 32). When the needle arm 58 is in the closed position, the needle is received within the distal end of the needle capture device 38 (FIG. 8). The needle capture device 38 is operated to securely engage the needle 70. The handle 24 is then operated to move the needle arm 58 toward the open position, thereby disengaging the needle arm 58 from the needle assembly 70, which remains in the needle capture device 38 (FIG. 33). The tissue retractor 40 is also released from the tissue and withdrawn back through the second catheter 36. The endoscope 12 is then moved to displace the cap assembly 50 relative to the sutured tissue 184. The needle 70 and suture 78 may be secured onto the tissue, such as by knotting or cinching, or the needle may be repositioned on the needle arm and additional suture loops may be formed within adjacent or other areas of tissue. Once the suturing is complete, the needle arm 58 is returned to a closed position, and the endoscope 12 and suturing device 22 are removed from the patient.

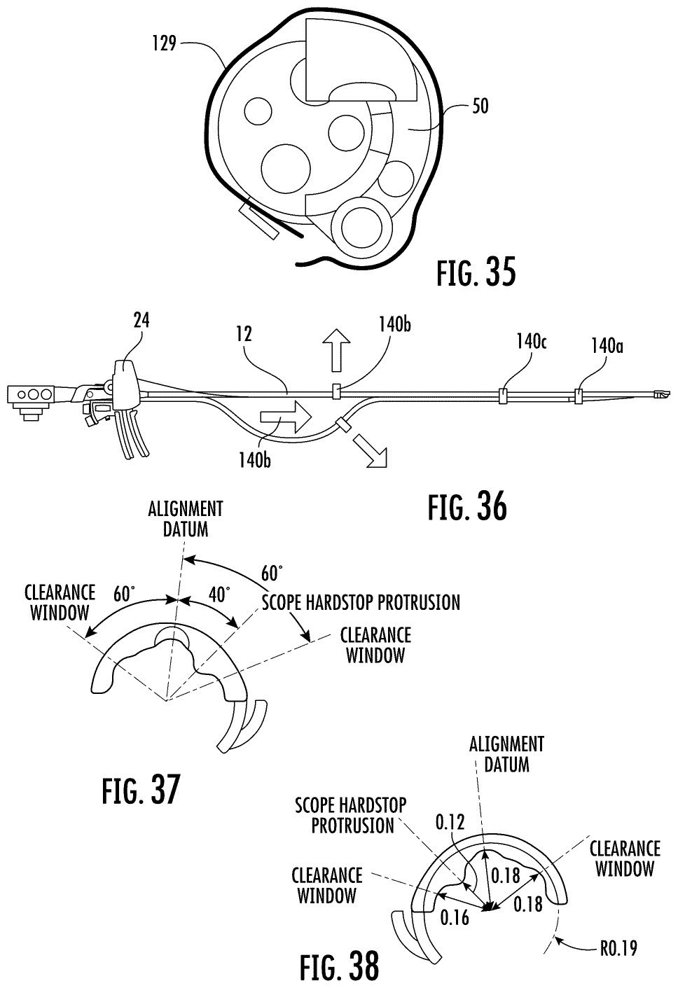

The suturing assembly is then released from over the endoscope by cutting, e.g., with a scissors 190, the two bands of tape 129 coupled to the hooks 120, 122 that secure the endoscope 12 relative to the cap assembly 50, and then unwrapping the tape 129 over the opening of the cap clip 102, as shown by both FIG. 34 and FIG. 35. Then, referring to FIG. 36, the tape strips 140b, 140c, 140a are broken apart, in turn, by applying force in the direction of arrow 174 to break each respective piece of tape at its perforation 152 (FIG. 17); as each tape strip was not directly adhered to the endoscope but only to itself and the sheath 89, the endoscope can be released once the strips are broken. The proximal handle 24 is then fully released from the endoscope 12.

In view of the above, in accordance with various principles of the present disclosure, an example of an embodiment of an endoscopic apparatus for use with an endoscope has a proximal handle assembly; a distal cap assembly; and a central portion adapted to extend along the endoscope between the handle assembly and the cap assembly. In some aspects, the handle assembly defines a recess in which the distal end of the endoscope is received. In some embodiments, the handle includes at least one hook at one side of the recess for receiving a tensile member that wraps around a portion of the cap assembly and the endoscope.

In some embodiments, the at least one hook is identified with a contrasting color relative to a majority of the cap assembly.

Additionally or alternatively, in some embodiments, the distal cap assembly includes a distal stop against which the distal end of the endoscope is intended to be abutted. In some embodiments, the distal stop includes indicia relative to which a feature of the endoscope is intended to be rotationally aligned. In some embodiments, the indicia include a contrasting color relative to a majority of the cap assembly. In some embodiments the indicia on the distal stop and the at least one hook are identified with a common color. In some embodiments, the feature of the endoscope with which the indicia are to be aligned is an instrument channel of the endoscope.

Additionally or alternatively, in some embodiments, the cap assembly is a suturing assembly including a movably mounted needle holder. In some aspects, operation of the proximal handle assembly moves the needle holder on the cap assembly.

In accordance with various principles of the present disclosure, an example of an embodiment of an endoscopic apparatus for use with an endoscope includes a proximal handle assembly; a distal cap assembly; and a central portion adapted to extend along the endoscope between the handle assembly and the cap assembly. In some aspects, the cap assembly defines a recess in which the distal end of the endoscope is received. Additionally or alternatively, the cap assembly includes two longitudinally spaced apart hooks at one side of the recess.

Additionally or alternatively, in some embodiments, each of the hooks are identified by a contrasting color relative to a color of a majority of the cap assembly.

Additionally or alternatively, in some embodiments, each of the hooks are identified by different hook indicia. In some embodiments, the hook indicia indicate an order of operation in which the hooks are intended to be used. In some embodiments, the hook indicia include numbers.

Additionally or alternatively, in some embodiments, the distal cap assembly includes a resilient clip that defines the recess.

Additionally or alternatively, in some embodiments the distal cap assembly includes a distal stop against which the distal end of the endoscope is intended to be abutted, the stop including contrasting alignment indicia relative to which a feature of the endoscope is intended to be rotationally aligned.

Additionally or alternatively, in some embodiments the distal cap assembly includes a distal stop against which the distal end of the endoscope is intended to be abutted, the stop including contrasting alignment indicia relative to which an instrument channel of the endoscope is intended to be rotationally aligned. In some embodiments the distal stop includes first and second recesses, and the alignment indicia is provided at the first recess.

In some embodiments, the cap assembly is a suturing assembly including a movably mounted needle holder, and operation of the proximal handle assembly moves the needle holder on the cap assembly.

In accordance with various principles of the present disclosure, an example of an embodiment of a securing system for use with an endoscope includes an endoscopic apparatus including a proximal handle assembly, a distal cap assembly; a central portion adapted to extend along the endoscope between the handle assembly and the cap assembly; and a spool of tape. In some embodiments, the distal cap assembly defines a recess in which the distal end of the endoscope is received and a hook at a side of the recess. In some embodiments, the spool of tape has a free end provided with a preformed loop adapted to be received over the hook. In some embodiments, the loop is adapted to be attached over the hook. In some embodiments, the tape is adapted to be wrapped about a portion of the cap assembly and the endoscope to couple the cap assembly and endoscope relative to each other.

In some embodiments, the system further includes a tape applicator including a handle and an extension with a spool mount. In some embodiments, the spool is coupled to the spool mount. In some embodiments, the spool is removably coupled to the spool mount.

In some embodiments, the cap assembly includes at least two longitudinally displaced hooks. In some embodiments, the securing system includes at least two spools of tape.

In some embodiments, the hook is identified by a contrasting color from a color of a majority of the cap assembly.

In some embodiments, the cap assembly includes at least two hooks. In some embodiments, each of the hooks is identified by different hook indicia. In some embodiments, the hook indicia identify an order of operation in which the hooks are intended to be used. In some embodiments, the hook indicia include a contrasting color from a majority of the cap assembly.

In some embodiments, the cap assembly is a suturing assembly including a movably mounted needle holder. In some embodiments, operation of the proximal handle assembly moves the needle holder on the cap assembly.

In accordance with various principles of the present disclosure, a method includes coupling a cap assembly of an endoscopic apparatus to an endoscope having a distal end. In some aspects, the cap assembly defines a recess in which the distal end of the endoscope is received. Additionally or alternatively, the cap assembly has first and second longitudinally displaced hooks at one side of the recess. In accordance with various principles of the present disclosure, the method includes inserting the distal end of the endoscope into the recess; providing a first length of tape having a first loop at a free end; coupling the first loop to the first hook; and wrapping at least a portion of the first length of tape about the cap assembly and the distal end of the endoscope.

In some aspects, the method further includes providing a second length of tape having a second loop at a free end; coupling the second loop to the second hook; and wrapping at least a portion of the second length of tape about the cap assembly and the distal end of the endoscope. In some aspects, the first hook is distally displaced from the second hook. In some aspects, the endoscopic apparatus includes a proximal handle and a central longitudinal sheath portion extending between the handle and the cap assembly. In some aspects, the central longitudinal portion includes a concave recess along its length. In some aspects, the method further includes inserting the endoscope into the recess of the central longitudinal sheath portion; and securing the central longitudinal sheath portion to the endoscope. In some aspects, the sheath portion is secured to the endoscope with a plurality of longitudinally spaced apart pieces of tape positioned at least partially circumferentially about the sheath portion and the endoscope. In some aspects, the central longitudinal sheath portion is crescent shaped and defines at least one lumen. In some aspects, at least one lumen includes a first lumen through which a flexible first catheter extends to the cap assembly, and a second lumen through which a flexible second catheter extends to the cap assembly. In some aspects, the cap assembly includes an actuation arm movably mounted thereon. In some aspects, the central longitudinal sheath portion includes a third lumen through which an actuation assembly extends. In some aspects, the actuation assembly is coupled between the proximal handle and the actuation arm and adapted for moving the actuation arm on the cap assembly upon operation of the proximal handle. In some aspects, the endoscopic apparatus includes a pull string having a proximal end and a distal end, the pull string extending from the proximal handle, through the central longitudinal sheath portion, and fixed to the cap assembly. In some aspects, the proximal end of the pull string is retracted relative to the proximal handle, and the distal end of the pull string is tensioned to cause the first and second catheters to flex and draw the cap assembly into retroflexion.

In some aspects, the cap assembly is a suturing assembly including a movably mounted needle holder.

In accordance with various principles of the present disclosure, an example of an embodiment of an endoscopic apparatus for use with an endoscope includes a proximal handle assembly; a distal cap assembly; a sheath portion; and a securing system. In some aspects, the cap assembly defines an assembly for positioning adjacent the distal end of the endoscope. In some aspects, the sheath portion defines a longitudinal recess. In some aspects, the sheath portion is adapted to extend along a portion of an exterior of the endoscope between the handle assembly and the cap assembly. In some aspects, the sheath portion is adapted to extend at least partially about the endoscope at the recess. In some aspects, the recess has a first side and a second side. In some aspects, the securing system is configured to secure the central portion to the exterior of the endoscope during in vivo operation of the endoscope. In some aspects, the securing system includes a plurality of longitudinally spaced apart lengths of tape pre-applied to the sheath portion on the first side of the recess. In some aspects, at least some of or each of the lengths of tape has an adhesive first portion at which the tape is adhered to the sheath on the first side of the recess, and a non-adhesive second portion extending from the first portion. In some aspects, at least some of or each of the lengths of tape has an adhesive third portion extending from the second portion and terminating in a free end.

In some aspects, the securing system further includes a removable non-adhesive protective strip over the third portion.

In some aspects, the protective strip extends into a tab with a free end protruding between the free end and the first portion.

Additionally or alternatively, the protective strip extends into an L-shaped non-adhesive tab. Additionally or alternatively, the tab has indicia indicating a direction in which the tab should be pulled to expose the third portion.

Additionally or alternatively, the protective strip has a contrasting color to the sheath portion.

In some aspects, each length of tape has a perforation between the, at, or near a junction of the second and third portions of the tape.

In some aspects, the securing system includes a first length of tape pre-applied at a distal end of the sheath portion, a second length of tape pre-applied at a central portion of the sheath portion, and a third length of tape pre-applied to the sheath portion between the first and second lengths of tape.

In some aspects, the sheath portion defines at least one lumen in a wall of the sheath portion.

In some aspects, the cap assembly is a suturing assembly including a movably mounted needle holder. In some aspects, operation of the proximal handle assembly moves the needle holder on the cap assembly.

In accordance with various principles of the present disclosure, a method of securing an endoscopic apparatus to an endoscope includes securing an endoscope having an external sheath to an endoscope. In some aspects, the endoscope has a circumference and a first length, and the sheath has a second length configured to extend about at least a portion of the circumference of the endoscope along at least a portion of the first length of the endoscope. In some aspects, the method includes providing the sheath with pre-applied portions of tape along second length; extending the sheath about the portion of the circumference of the endoscope along the first length; and wrapping the pre-applied portions of tape about a periphery of the endoscope and sheath to secure the sheath to the endoscope.

In some aspects, at least one or each pre-applied portion of tape includes an adhesive first portion at which the portion of tape is adhered to the sheath. Additionally or alternatively, at least one or each pre-applied portion of tape includes a non-adhesive second portion extending from the first portion. Additionally or alternatively, at least one or each pre-applied portion of tape includes an adhesive third portion extending from the second portion and terminating in a free end. Additionally or alternatively, at least one or each pre-applied portion of tape includes a removable protective strip over the third portion. In some aspects, the method further includes removing the removable protective strip from other the third portion, extending the second portion into contact with the endoscope, and adhering the third portion into contact with the sheath on an opposite side endoscope relative to the first portion.

In some aspects, the sheath defines a recess, the endoscope is inserted into the recess, and the first and third portions of the tape are located on opposite sides of the recess.

In some aspects, each pre-applied portion of tape includes a perforation. In some aspects, the method further includes releasing the endoscope from the sheath by applying a force to break the tape at the perforation. In some aspects, the tape is broken without cutting. In some aspects, the tape is broken by applying a force parallel to a longitudinal axis of the endoscope. In some aspects, the cap assembly is a suturing assembly including a movably mounted needle holder.

In accordance with various principles of the present disclosure, an example of an embodiment of an endoscopic apparatus for use with an endoscope includes a proximal handle assembly; a distal cap assembly; a sheath; a first catheter; a second catheter; and a transmission cable. In some aspects, the distal cap assembly is adapted to be positioned adjacent the distal end of the endoscope. In some aspects, the cap assembly has an end effector movable thereon. In some aspects, the sheath is a crescent-shaped flexible sheath. Additionally or alternatively, the sheath has a longitudinal side-opening recess sized to receive a portion of the endoscope between the handle assembly and the cap assembly. Additionally or alternatively, the sheath defines a first lumen, a second lumen, and a third lumen. In some aspects, the first catheter extends in and distally beyond the first lumen to the cap assembly. In some aspects, the second catheter extends in and distally beyond the second lumen to the cap assembly. In some aspects, the transmission cable extends from the handle assembly. In some aspects, the transmission assembly extends from the handle assembly into the third lumen. In some aspects, the transmission assembly extends distally beyond the third lumen to the cap assembly. In some aspects, the transmission assembly is operably coupled to the end effector. In some aspects, operation of the handle assembly moves the transmission cable to operate the end effector.

In some aspects, the cap assembly is a suturing assembly and the end effector is a needle holder.

In accordance with various principles of the present disclosure, an example of an embodiment of an endoscopic apparatus is configured for use with an endoscope having a proximal end and a distal end, an instrument channel, and a lens. In accordance with various principles of the present disclosure, the endoscopic apparatus includes a proximal handle; a distal cap assembly; and a central portion extending between the handle and cap assembly. In some aspects, the distal cap assembly defines an assembly for interacting with tissue. In some aspects, the distal cap assembly is adapted to be positioned adjacent the distal end of the endoscope. In some aspects, the distal cap assembly includes a clip to receive the distal end of the endoscope. In some aspects, the distal cap assembly includes a stop to limit distal travel of the endoscope. In some aspects, the stop has indicia against which a feature of the endoscope is rotationally aligned to ensure appropriate orientation of the lens and instrument channel of the endoscope without obstruction thereof. In some aspects, the central portion extends between the handle and cap assembly.

In some aspects, the stop is adapted to function with endoscopes from multiple manufacturers.

In some aspects, the cap assembly is a suturing assembly including a movably mounted needle holder. In some aspects, operation of the proximal handle moves the needle holder on the cap assembly.

In accordance with various principles of the present disclosure, an example of an embodiment of an endoscopic apparatus for use with an endoscope includes a proximal handle assembly; a distal cap assembly; a sheath; at least one catheter; and a pull string. In some aspects, the distal cap assembly is adapted to couple to the distal end of the endoscope spaced from the handle assembly by a first length. In some aspects, the sheath is a flexible sheath. In some aspects, the sheath has a proximal end and a distal end, the proximal end attached to the handle assembly. In some aspects, the sheath extends a second length less than the first length. In some aspects, the difference between the first length and the second length defines a gap. In some aspects, the sheath is adapted to extend at least partially about an exterior of the endoscope. In some aspects, the sheath defines at least one lumen. In some aspects, the at least one catheter extends through the at least one lumen from the proximal end of the sheath. In some aspects, the at least one catheter extends beyond the distal end of the sheath. In some aspects, the at least one catheter is fixed to the cap assembly. In some aspects, the pull string has a proximal end and a distal end. In some aspects, the pull string extends from the proximal end of the sheath, through the at least one lumen, out of the distal end of the sheath. In some aspects, the distal end of the pull string is fixed to the cap assembly. In some aspects, the proximal end of the pull string extends from an opening in the handle assembly. In some aspects, when the proximal end of the pull string is retracted relative to the handle, the distal end of the pull string is tensioned to cause the catheter to flex and draw the cap assembly into retroflexion across the gap.

In some aspects, the distal cap assembly is a suturing assembly including a needle movable relative to the at least one catheter. In some aspects, the pull string extends through a common lumen with a transmission cable for operating the suturing assembly.

In some aspects, the apparatus includes a pull handle attached to the proximal end of the pull string. In some aspects, the proximal handle assembly is adapted to releasably store the pull handle.

In some aspects, the apparatus further includes a locking system to temporarily retain the pull string under tension. In some aspects, the proximal handle assembly includes the locking system. In some aspects, the locking system includes a post having a seat and a washer provided over the post on the seat. In some aspects, the proximal end of the pull string is retained in position on the post. In some aspects, the proximal end of the pull string is retained in position on the post when wrapped around the post between the seat and the washer. In some aspects, the washer is made of a resilient material.

In accordance with various principles of the present disclosure, an example of an embodiment of an endoscopic system includes an endoscope and an external instrument channel device couplable to the endoscope. In some aspects, the endoscope has a proximal end and a distal end and an outer surface extending between the proximal and distal ends. In some aspects, the proximal end has a first handle. In some aspects, the distal end is adapted to retroflex upon actuation of the proximal end. In some aspects, the instrument channel device has a second handle. In some aspects, the instrument channel device has a sheath adapted to be coupled in close proximity to the outer surface of the endoscope between the proximal and distal ends. In some aspects, the instrument channel device has a cap removably coupled to the distal end of the endoscope. In some aspects, the instrument channel device has an instrument channel extending from the distal end of the sheath to the cap. In some aspects, the instrument channel device has a pull string extending from the second handle, through the sheath, and fixed relative to the cap. In some aspects, when the pull string is placed under tension, the cap is biased into a retroflex position about the instrument channel.

In some aspects, the cap is part of a suturing assembly.

In some aspects, the system further includes a pull handle attached to the proximal end of the pull string. In some aspects, the external instrument channel device includes a proximal handle assembly. In some aspects, the proximal handle assembly is adapted to releasably store the pull handle.

In some aspects, the system further includes a locking system to temporarily retain the pull string under tension. In some aspects, the external instrument channel device includes a proximal handle assembly, and the proximal handle assembly is provided with the locking system. In some aspects, the locking system includes a post having a seat and a washer provided over the post on the seat. In some aspects, the proximal end of the pull string is retained in position on the post. In some aspects, the proximal end of the pull string is retained in position on the post when wrapped around the post between the seat and the washer.

The suturing assemblies described above are adapted for use with an endoscope that does not necessarily have at least two instrument channels. As such, the suturing system can be used with smaller endoscopes that are available in many surgical settings and which can be more easily advanced through a natural orifice. In addition, as indicated aspects of the system can be used in other surgical treatment settings other than for suturing.

There have been described and illustrated herein embodiments of a suturing system as well as a surgical treatment system, as well as methods of using the same. While particular embodiments of the invention have been described, it is not intended that the invention be limited thereto, as it is intended that the invention be as broad in scope as the art will allow and that the specification be read likewise. Thus, while particular instruments and devices for advancement through the first and second lumen have been disclosed, it will be appreciated that other instruments can also be used through such lumen for like or even different purpose. Also, while the treatment system has been particularly described with respect to a cap assembly having an end effector in the form of a needle arm that carries a needle, it is recognized that alternatively one or more movable end effectors with other structure and purpose can be provided to the cap assembly. Also, while a tissue anchor in the form of a needle assembly has been described, the end effector can deploy different types of tissue anchors, including, e.g., clips. In addition, while a particular needle assembly has been described, other needle assemblies can similarly be used. Also, the size and instrument channel features of the endoscope with which the system is used is not critical, it is appreciated that various prior art systems cannot be properly used in a suturing operation in conjunction with endoscopes having fewer than two instrument channels, one for receiving a needle exchange device and the other for receiving a tissue retractor, whereas the present system is capable of complete operation without the provision of any channels through the endoscope. Further, while it is indicated that various features described herein are not limited to suturing applications, such as the retroflex system, it is specifically recognized that the retroflex system may be used in association with a cap assembly adapted to provide various other surgical applications including, but not limited to, staplers, clip appliers, band ligators, tissue manipulating instruments, cutting instruments, forceps, biopsy instruments, injection devices, as well as cap assemblies that have no significant function other than to support external catheters for passthrough of instruments. It will therefore be appreciated by those skilled in the art that yet other modifications could be made to the provided invention without deviating from its scope as claimed.

It is to be understood by one of ordinary skill in the art that the present discussion is a description of illustrative examples of embodiments only, and is not intended as limiting the broader aspects of the present disclosure.

All apparatuses and methods discussed herein are examples of apparatuses and/or methods implemented in accordance with one or more principles of this disclosure. These examples are not the only way to implement these principles but are merely examples, not intended as limiting the broader aspects of the present disclosure. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. It should be apparent to those of ordinary skill in the art that variations can be applied to the disclosed devices, systems, and/or methods, and/or to the sequence of steps of the method described herein without departing from the concept, spirit, and scope of the disclosure. It will be appreciated that various features described with respect to one embodiment typically may be applied to another embodiment, whether or not explicitly indicated. The various features hereinafter described may be used singly or in any combination thereof. Therefore, the present invention is not limited to only the embodiments specifically described herein, and all substitutes and modifications apparent to those skilled in the art are deemed to be within the spirit, scope, and concept of the disclosure as defined by the appended claims. Various further benefits of the various aspects, features, components, and structures of devices and systems such as described above, in addition to those discussed above, may be appreciated by those of ordinary skill in the art.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, engaged, joined, etc.) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A system for performing a medical procedure within a patient, said system comprising:
a flexible elongate member insertable to a target site within patient; and
a movable device movable with respect to said flexible elongate member;
wherein said movable device includes a reconfigurable portion which is reconfigurable by a user with respect to at least a portion of said flexible elongate member.

2. The system of claim 1, wherein said reconfigurable portion is configurable with respect to said flexible elongate member to maintain a selected position of said movable device with respect to said flexible elongate member.

3. The system of claim 2, wherein said reconfigurable portion is bendable or otherwise deformable with respect to said flexible elongate member.

4. The system of claim 1, wherein said flexible elongate member is a medical scope.

5. The system of claim 4, further comprising an auxiliary flexible tubular elongate member extendable along said medical scope and defining a lumen through which said movable device is movable.

6. The system of claim 4, wherein said movable device is movable through a working channel defined through said medical scope.

7. The system of claim 1, wherein said movable device includes an operable component on a distal end thereof configured to engage tissue, wherein tissue engaged by said operable component exerts a distally-directed force on said movable device.

8. The system of claim 7, wherein said reconfigurable portion maintains said movable device with respect to said flexible elongate member and against a distally-directed force exerted thereon by tissue engaged by said movable device.

9. The system of claim 7, wherein said operable component is a helical tissue engagement element.

10. The system of claim 1, wherein said reconfigurable portion is adjacent a user-engageable portion of said movable device.

11. The system of claim 1, further comprising an additional device operable with said movable device.

12. The system of claim 11, wherein said additional device is a suturing device and said movable device comprise a tissue-engagement element.

13. A system for performing a medical procedure within a patient, said system comprising:
a movable device movable with respect to another component of said system;
wherein said movable device comprises:
a reconfigurable portion which is reconfigurable by a user with respect to at least said other component of said system; and
an operable component comprising a tissue engagement element.

14. The system of claim 13, wherein said tissue engagement element comprises a helical tissue-engaging element formed from a laser-cut tube.

15. The system of claim 13, wherein said movable device comprises a flexible control wire, at least a proximal portion thereof forming said reconfigurable portion of said movable device.

16. The system of claim 15, wherein said operable component comprises a tissue-engagement element coupled to a distal end of said flexible control wire.

17. The system of claim 13, further comprising an endoscope and a suturing device operatively associated with said endoscope, said movable device being movable with respect to said suturing device to draw tissue proximally into a path of a needle of said suturing device.

18. A method of operating a system configured to perform a procedure within a patient, said method comprising:
moving a first device with respect to a component of the system; and
reconfiguring a reconfigurable portion of the first device with respect to the component of the system to maintain the position of the first device in a selected position.

19. The method of claim 18, wherein:
the first device is a tissue engagement element and the component is a suturing device;
moving the first device further comprises advancing the tissue engagement element into tissue, and retracting the tissue engagement element to draw the tissue in the path of the suturing device; and
reconfiguring the reconfigurable portion of the first device includes engaging the reconfigurable portion with respect to a component of the system to maintain the position of the first device to maintain tissue in the path of the suturing device.

20. The method of claim 19, further comprising reconfiguring the reconfigurable portion of the first device to release the movable device from its position engaged with a component of the system.
